(19) United States
(12) Patent Application Publication
(10) Pub. No.: US 2021/0289791 A1
(43) Pub. Date: Sep. 23, 2021

(54) BIOPOLYMER-COATED TWO-DIMENSIONAL TRANSITION METAL CHALCOGENIDES HAVING POTENT ANTIMICROBIAL ACTIVITY

(71) Applicant: ARIZONA BOARD OF REGENTS ON BEHALF OF ARIZONA STATE UNIVERSITY, Scottsdale, AZ (US)

(72) Inventors: Alexander Green, Scottsdale, AZ (US); Qing Hua Wang, Scottsdale, AZ (US); Sanchari Saha, Tempe, AZ (US); Abhishek Debnath, Tempe, AZ (US)

(21) Appl. No.: 17/264,766

(22) PCT Filed: Jul. 30, 2019
(86) PCT No.: PCT/US2019/044183

§ 371 Date: Jan. 29, 2021

Related U.S. Application Data
(60) Provisional application No. 62/711,998, filed on Jul. 30, 2018.

(51) Int. Cl. A01N 59/16; A01N 59/02; A01N 25/32; A01N 25/10; A61L 31/16; A61L 31/10

(52) U.S. Cl. CPC A01N 59/16; A01N 59/02; A01N 25/32; A01N 25/10; A61L 2400/12; A61L 31/10; A61L 2300/102; A61L 2300/258; A61L 2300/404; A61L 31/16

(57) ABSTRACT

Biocompatible polymer-coated transition metal chalcogenide (TMC) nanomaterials are provided herein. In particular, provided herein are two-dimensional polymer-coated TMC nanomaterials having excellent antimicrobial properties and biocompatibility, as well as methods of inhibiting microbiological growth on, or in, devices coated by or otherwise comprising the biocompatible polymer-coated transition metal chalcogenide (TMC) nanomaterials. In some cases, the biopolymer coating encapsulating the TMC nanomaterial comprises short synthetic single-stranded DNAs (ssDNAs). As described herein, ssDNA-encapsulated TMDCs exhibit no cytotoxicity against human cell lines at concentrations up to 0.25 mg/mL, but they exhibit exceptionally strong bactericidal activity against both gram-positive and gram-negative bacteria, including antibiotic-resistant Escherichia coli and a gram-positive methicillin-resistant Staphylococcus aureus (MRSA) strain. In other cases, TMDCs encapsulated by poly-L-lysine and Pluronic F77 display strong activity against multi drug resistance bacteria and form coatings that strongly inhibit bacterial biofilms, while TMDCs encapsulated by chitosan exhibit strong activity against fungi.

### CROSS-REFERENCE TO RELATED APPLICATIONS

This application is a national stage filing under 35 U.S.C. § 371 of International Application No. PCT/US2019/044183, filed Jul. 30, 2019, which claims priority to U.S. Patent Application No. 62/711,998, filed Jul. 30, 2018, each of which are incorporated by reference herein as if set forth in their entirety.

### STATEMENT REGARDING FEDERALLY SPONSORED RESEARCH

Not applicable.

### REFERENCE TO A SEQUENCE LISTING SUBMITTED VIA EFS-WEB

The content of the ASCII text file of the sequence listing named “112624-01232_Sequence_Listing.TXT” which is 1,946 BYTES in size was created on Jan. 28, 2021, and electronically submitted via EFS-Web herewith the application is incorporated herein by reference in its entirety.

### BACKGROUND

Antibiotic drug resistance is recognized as one of the most pressing threats to global health. Spurred by the misuse and overuse of commonly prescribed antibiotics, drug-resistant bacteria are becoming increasingly common and infections that were once easily treated can now force patients into extended hospital stays. In the United States alone, infections by drug-resistant bacteria have been estimated to cause 2 million serious infections and cause 23,000 deaths each year, leading to billions of dollars in societal and economic costs. Despite this alarming threat, the number of antibiotics under development remains low raising the possibility of rampant drug resistance reversing many of the advances of modern medicine. This global health challenge thus demands new approaches to combatting and eradicating bacterial infections.

Two-dimensional (2D) materials have emerged as promising antibacterial materials owing to their high surface areas and atomically thin edges, which can promote strong interactions with bacterial cell walls, and in some cases the ability to generate redox species to trigger cell death. Of these materials, graphene and its close relative graphene oxide have been most extensively studied and provided varying activity depending on the preparation method. Transition metal dichalcogenides (TMDCs), which have been shown to have better biocompatibility than graphene and can be enzymatically degraded, have also been studied for antibacterial activity. Chemically exfoliated MoS₂, for instance, successfully killed 93.4% of Escherichia coli cells upon exposure at 80 μg mL⁻¹, while the same concentration of WSe₂ coated by long salmon-derived DNA eliminated only 82.3% of the same bacteria. Hydrothermally synthesized WS₂ was shown to kill 98.67% and 99.98% of E. coli and Bacillus subtilis cells, respectively, but only at very high concentrations of 250 μg mL⁻¹. Despite their clear antibacterial activity, these 2D TMDCs failed to match the activity of common antibiotics, which can eliminate >99.999% of cells at similar concentrations. To obtain comparable levels of activity, 2D TMDCs have required more aggressive conditions, extended treatment times, and light-driven reactions. WS₂ dispersed in the surfactant solutions completely eliminated cultures of E. coli and Staphylococcus aureus, yet it exhibited significant toxicity toward human cell lines. Liu et al. (Nature Nanotechnology 11:1098-1104 (2016)) demonstrated a water disinfection system that employed vertically aligned MoS₂ layers and visible light to eliminate >99.999% of E. coli and Enterococcus faecalis cells; however, this light-driven mechanism would be challenging to apply to bacterial infections. Accordingly, there remains a need in the art for innovative materials that exhibit exceptional antimicrobial activity against bacteria and fungi, including multi-drug resistant (MDR) organisms.

### SUMMARY

Provided herein are biocompatible polymer-coated transition metal chalcogenide (TMC) nanomaterials that address and surpass the limitations of previously described antimicrobial materials. In particular, provided herein are two-dimensional polymer-coated TMC nanomaterials having excellent antimicrobial properties and biocompatibility, as well as methods of inhibiting microbiological growth on, or in, devices coated by or otherwise comprising the biocompatible polymer-coated transition metal chalcogenide (TMC) nanomaterials.

In a first aspect, provided herein is a biocompatible polymer-coated transition metal chalcogenide (TMC) nanomaterial comprises or consisting essentially of a two-dimensional dispersion of TMC at least partially coated with a biocompatible polymer. The TMC can be a transition metal dichalcogenide, transition metal monochalcogenide, transition metal trichalcogenide, post-transition metal monochalcogenide, or post-transition metal trichalcogenide. The TMC can be selected from MoS₂, MoSe₂, WS₂, WSe₂, Bi₂Se₃, Bi₂S₃, SnSe, and SnS. The biocompatible polymer can be selected from a single-stranded DNA (ssDNA), a single-stranded RNA (ssRNA), peptide, poly-L-lysine, poly-D-lysine, Pluronic polymers, Tetronic polymers, and chitosan, or a combination thereof.

In another aspect, provided herein is a method for inhibiting microbiological growth on, or in, a medium. The method can comprise or consist essentially of contacting the medium with a biocompatible polymer coated TMC nanomaterial as described herein. Contacting the medium with the biocompatible polymer coated TMC nanomaterial can inhibit growth of one or more multidrug resistant (MDR) microbes.

In a further aspect, provided herein is a method for preparing a biocompatible polymer coated transition metal chalcogenides (TMC). The method can comprise or consist essentially of (a) ultrasonicating a bulk TMDC powder in an aqueous solutions comprising a biocompatible polymer, whereby an ultrasonicated TMDC solution is obtained; (b) centrifuging the ultrasonicated TMDC solution, whereby a supernatant and a precipitate are obtained; and (c) collecting the supernatant which comprises a two-dimensional dispersion of biocompatible polymer-coated TMDC. The TMC can be a transition metal dichalcogenide, transition metal monochalcogenide, transition metal trichalcogenide, post-transition metal monochalcogenide, or post-transition metal trichalcogenide. The biocompatible polymer can be selected from a single-stranded DNA (ssDNA), a single-stranded RNA (ssRNA), peptide, poly-L-lysine, poly-D-lysine, Pluronic polymers, Tetronic polymers, and chitosan, or a combination thereof. The biocompatible polymer can be ssDNA or ssRNA having a length of 10 nucleotides to 80 nucleotides. The ssDNA can have a nucleotide sequence comprising at least ten repeats of GT (GT₁₀) (SEQ ID NO: 1). The ssDNA can have a nucleotide sequence comprising 10-20 consecutive thymidine (T) bases.

In another aspect, provided herein is a method for inhibiting microbiological growth on, or in, a medium which comprises coating the medium with a biocompatible polymer coated TMC nanomaterial prepared according to any of the methods provided herein. The medium can be a medical device. Coating the medium with the biocompatible polymer coated TMC nanomaterial can inhibit growth of one or more multidrug resistant (MDR) microbes.

### BRIEF DESCRIPTION OF THE DRAWINGS

The present invention will be better understood and features, aspects, and advantages other than those set forth above will become apparent when consideration is given to the following detailed description thereof. Such detailed description makes reference to the following drawings, wherein:

FIGS. 1A-1D demonstrate dispersion of two-dimensional transition metal dichalcogenides in ssDNA. a, Schematic illustration showing encapsulation of 2D TMDCs with short ssDNAs. b, Photograph of vials of the four different 2D TMDCs dispersed in ssDNA sequence T₂₀, and showing the differences in dispersion efficiency for MoSe₂ by T₂₀ (SEQ ID NO: 2) and (CA)₁₀. (SEQ ID NO: 3) c, UV-vis absorption spectra of DNA dispersed TMDCs, with excitonic peaks indicated by (*) symbols. d, Plots of concentrations of TMDC dispersions as a function of DNA sequence, from left to right ss DNA sequences are A₂₀ (SEQ ID NO: 4), T₂₀ (SEQ ID NO: 2), C₂₀ (SEQ ID NO: 5), (GGGGA)₄ (SEQ ID NO: 6), (GGGGT)₄ (SEQ ID NO: 7), (GT)₁₀ (SEQ ID NO: 1), (CA)₁₀ (SEQ ID NO: 3). The dispersions shown in panel (b) are indicated by (*).

FIGS. 2A-2L demonstrate microscopic analysis of TMDC dispersions. Left column: TEM images demonstrating successful production of two-dimensional MoS₂, MoSe₂, WS₂ and WSe₂ nanosheets. Middle column: HRTEM images of atomic structures of nanosheets (insets: electron diffraction patterns showing crystallinity). Right column: AFM images of nanosheets deposited from ssDNA dispersions onto HOPG substrates by spin coating. Insets: Height profiles along the dashed lines across each nanosheet.

FIGS. 3A-3G show antibacterial efficiency of MoSe₂/ssDNA. a, Antibacterial efficiency of MoSe₂/T₂₀ (SEQ ID NO: 2) compared to graphene on model gram-positive S. aureus. b, Antibacterial efficiency of MoSe₂/T₂₀ (SEQ ID NO: 2) compared to graphene on model gram-negative E. coli MG1655. c, Antibacterial efficiency of MoSe₂/T₂₀ (SEQ ID NO: 2) compared to MoSe₂ dispersed with Pluronic F77 and salmon genomic dsDNA on S. aureus. d, Antibacterial efficiency of MoSe₂/T₂₀ (SEQ ID NO: 2) compared to MoSe₂ dispersed with Pluronic F77 and salmon genomic dsDNA against E. coli. e, Antibacterial activity of MoSe₂/T₂₀ (SEQ ID NO: 2) against three MDR E. coli strains. f, Antibacterial activity of MoSe₂/T₂₀ against gram-positive E. faecalis and MRSA strains. g, Antibacterial activity of MoSe₂/T₂₀ (SEQ ID NO: 2) against gram-negative K. pneumoniae, A. baumannii, P. aeruginosa, and E. cloacae strains.

FIGS. 4A-4F demonstrate morphological changes of bacteria after exposure to MoSe₂/ssDNA. a, Images of untreated MRSA sample under SEM (left images) and TEM (right images) at lower (top images) and higher (bottom images) magnifications. b, Images of MRSA treated with 40 μg ml⁻¹ of MoSe₂/T₂₀ (SEQ ID NO: 2). c, SEM and TEM images of MRSA treated with 80 μg ml⁻¹ of MoSe₂/T₂₀ (SEQ ID NO: 2). d-f, SEM and TEM images of A. baumannii not treated (d) and treated with 40 μg ml⁻¹ of MoSe₂/T₂₀ (SEQ ID NO: 2) (e), and 80 μg ml⁻¹ of MoSe₂/T₂₀ (SEQ ID NO: 2) (f).

FIGS. 5A-5E demonstrate mechanistic analysis of antibacterial action of MoSe₂/T₂₀ (SEQ ID NO: 2). a-b, Change in membrane potential after interactions of MoSe₂/T₂₀ (SEQ ID NO: 2) with S. aureus (a) and E. coli (b). c-d, Oxidative stress generated in S. aureus (c) and E. coli (d) after interactions with MoSe₂/T₂₀ (SEQ ID NO: 2) (e) Fold change in mRNA expression after interactions with MoSe₂/T₂₀ (SEQ ID NO: 2) in E. coli.

FIG. 6 is a schematic illustration of protocol for exfoliation of bulk MoSe₂ in 0.5% chitosan (CS) solution.

FIGS. 7A-7C demonstrate characterization of MoSe₂/CS nanosheets. (A) Glass vial containing dark brown MoSe₂/CS solution. (B) UV-vis of MoSe₂/CS having characteristic peaks (*) at 700 nm and 800 nm. (C) TEM images showing MoSe₂/CS nanosheets with lateral dimension to be −70 nm by −200 nm.

FIGS. 8A-8D demonstrate antifungal efficiency of MoSe₂/CS against unicellular and filamentous fungi. (A) MFC of BSL-1 I. orientalis, S. cerevisiae, and C. parapsilosis was determined to be 12.5 μg ml⁻¹ determined using the help of microdilution method. (B) MFC of BSL-2 C. albicans, C. neoformans, and C. gattii was determined to be 50 μg ml⁻¹. (C, D) MIC of unicellular C. albicans and filamentous A. fumigatus was tested for 16 h and 24 h to be 12.5 μg ml⁻¹ and 32.5 μg ml⁻¹ respectively.

FIGS. 9A-9H demonstrate multimodal killing mechanism of MoSe₂/CS against C. albicans and A. fumigatus. (A, C) SEM images of healthy control cells of C. albicans (A) and A. fumigatus (C). (B, D) SEM images showing disruptive features (red), morphological deformation (blue) and broken outer membrane (green) of C. albicans (B) and A. fumigatus (D) in the presence of MoSe₂/CS respectively. (E, G) TEM images of control cells with intact cytoplasm of C. albicans (E) and A. fumigatus (G). (F, H) TEM images of Sharp edges of MoSe₂ flakes interacting with cell wall (pink) leading to rupturing of cell wall (green) and cytoplasmic leakage (orange) C. albicans (F) and A. fumigatus (H) in the presence of MoSe₂/CS respectively.

FIGS. 10A-10C demonstrate biocompatibility data for a MoSe₂/CS solution. (A) Hemolysis assay to determine the toxicity of MoSe₂/CS and 0.5% CS alone against RBCs. (B) Viability of HEK 293 mammalian cells tested with the alamarBlue assay in the presence of MoSe₂/CS and 0.5% CS alone. (C) Percent cytotoxicity generated by MoSe₂/CS and 0.5% CS alone against HEK 293 cells.

FIGS. 11A-11B present (a) a schematic showing the preparation of MoSe₂ in presence of cationic polypeptide poly-L-lysine and Pluronic F77; and (b) transmission electron microscopy demonstrating exfoliated MoSe₂ in presence of cationic Poly-L-Lysine and Pluronic F77.

FIGS. 12A-12C present characterization of MoSe₂/PLL/F77 nanosheets. (A) Glass vial containing MoSe₂/PLL/F77 has a dark brown color. (B) UV-vis of MoSe₂/PLL/F77 having characteristic peaks at 700 nm and 800 nm. (C) TEM images showing MoSe₂/PLL/F77 nanosheets with lateral dimension to be ˜50 nm by ˜150 nm.

FIGS. 13A-13D demonstrate quantitative measurement of biofilm eradication in presence of MoSe₂/PLL/F77. (A) MBC of MRSA, Acinetobacter baumannii (A. baumannii) and Pseudomonas aeruginosa (P. aeruginosa) in solution. (B) MBEC of MRSA, A. baumannii and P. aeruginosa biofilm. (C) Crystal violet assay to determine the relative biofilm mass left in presence of MoSe₂/PLL/F77. (D) XTT assay to determine the relative cell viability in presence of MoSe₂/PLL/F77.

FIGS. 14A-14N demonstrate qualitative analysis of effects of MoSe₂/PLL/F77 on biofilm. (A-C) Confocal images of the MRSA (A), A. baumannii (B), and P. aeruginosa (C) untreated control films. (D-F) Confocal images of MRSA (A), A. baumannii (B), and P. aeruginosa (C) biofilms treated with ˜150 μg ml⁻¹ of MoSe₂/PLL/F77. (G) Percent of live cells present in control samples in comparison to treated samples. (H-J) SEM images of MRSA (H), A. baumannii (I), and P. aeruginosa (J) cells in absence of MoSe₂/PLL/F77. (K-M) SEM images of MRSA (K), A. baumannii (L), and P. aeruginosa (M) treated with ˜150 μg ml⁻¹ of MoSe₂/PLL/F77. (N) Comparison number of cells present in the control samples versus the treated samples of each strain.

FIGS. 15A-15I demonstrate analysis of biofilm growth against MoSe₂/PLL/F77 coating on different surfaces. (A, B) Biofilm growth of MRSA, A. baumannii, and P. aeruginosa on an uncoated (A) and coated (B) PMMA surface respectively. (C) Comparing number of cells present on an uncoated and coated surface of PMMA coated glass slides. (D, E) Biofilm growth of MRSA, A. baumannii, and P. aeruginosa on uncoated (D) and coated (E) hydrophilic PTFE surface. (F) Comparing number of cells present on uncoated and coated surface of hydrophilic PTFE membrane. (G, H) Biofilm growth of MRSA, A. baumannii, and P. aeruginosa on uncoated (G) and coated (H) medical grade Ti-alloy. (I) Comparing number of cells present on coated and uncoated surface of medical grade Ti-alloy.

FIGS. 16A-16K demonstrate analysis of MoSe₂/PLL/F77 coating on the same MBEC stub. (A, B, C) MRSA (A), A. baumannii (B), and P. aeruginosa (C) biofilm growth on top portion of MBEC stub that is uncoated. (D, E, F) Lack of biofilm growth of MRSA (D), A. baumannii (E), and P. aeruginosa (F) on the bottom region of the same MBEC stub that is coated with MoSe₂/PLL/F77. (G, H, I) EDX analysis of MBEC stub partially coated with MoSe₂/PLL/F77 showing the presence and absence of MoSe₂ on coated and uncoated region with MRSA (G), A. baumannii (H), and P. aeruginosa (I) biofilm grown on it. (J) Bar plot displaying the difference between the number of bacteria cells present on the uncoated and coated region of the same stub. (K) The difference in atomic percentage of each element displayed on the uncoated and coated region of the same stub.

FIGS. 17A-17B demonstrate biocompatibility data for a MoSe₂/PLL/F77 coating. (A) Viability of HEK 293 mammalian cells tested with the help alamarBlue assay in presence of MoSe₂/PLL/F77 coated hydrophilic PTFE membrane. (B) Percent cytotoxicity generated by MoSe₂/PLL/F77 coated hydrophilic PTFE membrane on HEK 293 cells.

While the present invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the appended claims.

### DETAILED DESCRIPTION OF THE DISCLOSURE

All publications, including but not limited to patents and patent applications, cited in this specification are herein incorporated by reference as though set forth in their entirety in the present application.

Two-dimensional transition metal dichalcogenides (TMDCs) have been widely explored for a range of biomedical applications such as drug delivery, tissue engineering, and tomographic imaging. Previous reports on solution-phase processing of TMDCs have relied on aggressive lithiation treatments, which require toxic chemicals and in turn hamper their use in biological systems, or have required amphiphilic surfactants to encapsulate the hydrophobic TMDCs, which can reduce biocompatibility. Provided herein is an innovative biopolymer-based strategy for solution-phase processing of TMDCs that employs short synthetic single-stranded DNAs (ssDNAs) to stably encapsulate TMDC nanosheets in aqueous solution. ssDNA-assisted exfoliation yields TMDC dispersions with concentrations up to 1 mg/mL and enables a variety of functional chemical moieties to be attached to the TMDC surface through versatile synthetic DNA chemistries. Also provided herein is an innovative preparation of 2D TMDCs having remarkable antimicrobial activity against multidrug resistant (MDR) strains of gram-negative E. coli and gram-positive S. aureus yet do not exhibit toxicity against human cells, thus overcoming problems associated with conventional TMDCs. As described herein, 2D TMDCs encapsulated in short sequences of single-stranded DNA (ssDNA) do not exhibit toxicity against human cells, yet are capable of complete elimination of MDR strains at concentrations as low as 80 μg mL¹. Moreover, through direct comparisons, it was determined that short ssDNA-encapsulated MoSe₂ nanosheets provide substantially higher antibacterial activity than widely studied graphene oxide and other preparations of 2D TDMCs.

Accordingly, embodiments described herein relate to polymer coated 2D transition metal dichalcogenides and methods of using such materials to treat infections caused by microbial organisms including multidrug resistant bacteria and other human pathogenic microbes. The materials provided herein are useful as antimicrobials against drug resistant microbes. As the materials provided herein exhibit no or minimal toxicity to human cells, the materials may be incorporated into coatings having strong antimicrobial activity, such as those used in hospitals for passive disinfection purposes. The formulations described herein are ultrathin materials that exhibit exceptional antimicrobial activity against microbial organisms such as bacteria and fungi. These ultrathin materials can eradicate multidrug resistant strains of microbial organisms at reasonable concentrations. Without being bound to any particular theory or mode of action, it is believed that sharp knife-like edges of ssDNA-coated TMDC flakes disrupt bacterial cell membranes to induce cytoplasmic leakage and, thus, exert antimicrobial effects by multimodal mechanism of action that make the development of future resistance challenging.

Compositions

In a first aspect, provided herein are transition metal chalcogenide (TMC) nanomaterials (e.g., two-dimensional (2D) sheets) in which the TMCs are coated with a biocompatible polymer, where the coated TMC nanomaterials exhibit antimicrobial activity. As described herein, biocompatible polymer coatings having known antimicrobial activity exhibit surprisingly enhanced antimicrobial activity when used in combination with 2D TMCs as described herein.

As used herein, the term “transition metal chalcogenide” (“TMC”) refers to an ultrathin material comprising metal compounds that comprise at least one chalcogen anion and at least one more electropositive element (e.g., sulfides, selenides and tellurides), and encompasses transition metal dichalcogenides (TMDCs), transition metal monochalcogenides, transition metal trichalcogenides, and several post-transition metal chalcogenides (PTMCs) (e.g., post-transition metal monochalcogenide, post-transition metal trichalcogenide). As used herein, the term “transition metal dichalcogenide” refers to TMCs having the formula ME₂, where M=a transition metal (or in some cases a post-transition metal) and E=sulfide (S), selenide (Se), telluride (Te). Exemplary transition metal dichalcogenides include, without limitation, molybdenum disulfide (MoS₂), molybdenum diselenide (MoSe₂), titanium disulfide (TiSe₂), tungsten diselenide (WSe₂), and tungsten disulfide (WS₂). Transition metals include, without limitation, molybdenum (Mo), tungsten (W), titanium (Ti), iron (Fe), cobalt (co), nickel (Ni), zinc (Zn), copper (Cu), zirconium (Zr), hafnium (Hf), vanadium (V), niobium (Nb), tantalum (Ta), technetium (Tc), and rhenium (Re). Post-transition metals include, without limitation, bismuth (Bi), antimony (Sb), and tin (Sn). In some cases, the TMC is selected from MoS₂, MoSe₂, WS₂, WSe₂, Bi₂Se₃, Bi₂S₃, Bi₂Te₃, Sb₂Se₃, Sb₂S₃, Sb₂Te₃, SnSe, and SnS.

As used herein, the terms “nanomaterial” refers to ultrathin, nanoscale materials. Nanomaterials can be nanosheets, nanoparticles, nanotubes, or other nanoscale structures. The term “nanosheet” as used herein refers to ultrathin, two-dimensional nanoscale materials. Such nanosheets can be thousands of square nanometers in lateral area but have thicknesses below approximately 15 nanometers and can consist of only a few atomic layers or a single atomic layer. In some cases, lateral sizes of the nanosheets are about 10 nm by about 10 nm, about 20 nm by about 20 nm, about 50 nm by about 50 nm, about 100 nm by about 100 nm, about 200 nm by about 150 nm, about 250 nm by about 200 nm, about 300 nm by about 200 nm, about 250 nm by about 250 nm, about 350 nm by about 200 nm, or about 500 nm by about 500 nm.

As used herein, the term “polymer” refers to macromolecules comprising a combination of many small units (monomers) that repeat themselves along the long chain. Polymers can be produced by polymerization of one or more monomers, used to impart desirable chemical, physical, and biological properties to the resulting material. Biocompatibility refers to the ability of a biomaterial to perform its desired function with respect to a medical therapy, without eliciting any undesirable local or systemic effects in the recipient or beneficiary of that therapy, but generating the most appropriate beneficial cellular or tissue response in that specific situation, and optimizing the clinically relevant performance of that therapy. With respect to biocompatible materials, the polymeric material should have known toxicological properties and exhibit sufficiently inert properties when in contact with blood and tissue. Biocompatible polymers suitable for the materials and methods provided herein include, without limitation, single-stranded nucleic acids (e.g., DNA, RNA, siRNA), single-stranded DNA (ssDNA), single-stranded RNA (ssRNA), poly-L-lysine, poly-D-lysine, Pluronic polymers, Tetronic polymers, peptides, chitosan, hyaluronic acid (HA), dextran, polyhydroxyalkanoates (PHAs), hydroxypropyl methylcellulose, hydroxypropyl cellulose, methyl cellulose, carboxymethyl cellulose, ethyl cellulose, and ethyl methyl cellulose, and combinations thereof.

In certain embodiments, the biocompatible polymer is a nucleic acid such as a single-stranded DNA (ssDNA) molecule or a single-stranded RNA (ssRNA) molecule. Preferably, the polymer is a synthetic ssDNA or ssRNA molecule having a sequence comprising repeats of particular nucleobases or pairs of nucleobases. In some cases, ssDNAs or ssRNAs have a length of 5 nucleotides to about 80 nucleotides (e.g., 5-nt, 10-nt, 15-nt, 20-nt, 25-nt, 30-nt, 35-nt, 40-nt, 45-nt, 50-nt, 55-nt, 60-nt, 65-nt, 70-nt, 75-nt, 80-nt lengths, inclusive). Preferably, ssDNAs or ssRNAs useful for the coated TMCs of this disclosure have a length of about 10-nt, 20-nt, or 40-nt. In some cases, ssDNAs or ssRNAs useful for the coated TMCs of this disclosure can have any sequence up to about 80 bases. In some cases, the sequence comprises repeated bases or pairs of bases, where by the bases are thymidine (T), guanine (G), cytosine (C), and adenosine (A). Since guanine-rich ssDNAs having lengths of 20-nt and greater are hard to synthesize, it may be helpful to synthetize ssDNAs comprising four or more repeats of GGGGA or GGGGT.

As described herein, methods for preparing two-dimensional transition metal chalcogenides coated with biocompatible polymers involve a biopolymer-based strategy for solution-phase processing of TMCs in which TMC nanosheets in aqueous solution are stably encapsulated in nucleic acids, poly-L-lysine, chitosan, or other biocompatible polymers.

In some cases, coated 2D TMCs of this disclosure are prepared according to the following steps: ultrasonicating a bulk TMDC powder in an aqueous solutions comprising synthetic ssDNA, whereby an ultrasonicated TMDC solution is obtained; centrifuging the ultrasonicated TMDC solution, whereby a supernatant and a precipitate are obtained; and collecting the supernatant which comprises two-dimensional dispersions of ssDNA-coated TMDC.

In some cases, the methods further comprise attaching one or more functional chemical moieties to the TMC surface through versatile synthetic DNA chemistries. For example, one or more proteins can be tethered to ssDNA-coated TMC nanomaterials using well-established DNA oligonucleotide chemistries. Methods for covalent attachment of proteins to nucleic acids include, without limitation, chemical cross-linking of oligonucleotides to protein lysine or cysteine residues (e.g., thiol chemistry), expressed protein-ligation, chemoenzymatic methods, and the use of photoaptamers.

Nucleic acids and/or other moieties of the invention may be isolated. As used herein, “isolated” means separate from at least some of the components with which it is usually associated whether it is derived from a naturally occurring source or made synthetically, in whole or in part.

Nucleic acids and/or other moieties of the invention may be purified. As used herein, purified means separate from the majority of other compounds or entities. A compound or moiety may be partially purified or substantially purified. Purity may be denoted by a weight by weight measure and may be determined using a variety of analytical techniques such as but not limited to mass spectrometry, HPLC, etc.

Methods of Use

According to another aspect, provided herein are methods of killing a microorganism or inhibiting microbiological growth on a surface or in a medium by the application of an effective amount of a composition of this disclosure to the medium. In some cases, compositions of this disclosure are used as antimicrobial surface coatings for healthcare-associated products, medical devices and components, medical electronics, products used in dental and veterinary fields, products used in food preparation or food storage, and other products for which the prevention of bacterial and/or fungal growth and spread is important. In such cases, the method comprises coating the medium (healthcare-associated product, medical device, etc.) with an effective amount of biopolymer-coated TMC of this disclosure. As used herein, the term “coating” refers to attaching or depositing, by any suitable process, a composition of this disclosure onto a medium (e.g., healthcare-associated product, medical device, etc.) such that the deposited composition covers across some or all outer surface areas of the medium. In some cases, coating comprises covering, at least partially, outer surface areas of the medium as well as one or more interior surfaces (e.g., an internal component of a medical device). Coating of a medium does not have to be complete. In particular, it is preferable in some cases to provide composition to only a portion or some portions of the item to be coated, thus resulting in an item that is at least partially coated by a composition of this disclosure. In some cases, a coating includes one or more coating layers. A coating can have a substantially constant or a varied thickness.

As used herein, the terms “antimicrobial,” “microbicidal,” or “biocidal” refer to the ability to kill at least some types of microorganisms, or to inhibit the growth or reproduction of at least some types of microorganisms. The compositions prepared in accordance with the present disclosure have microbicidal activity (antimicrobial) against a broad spectrum of pathogenic microbial organisms, also known as microorganisms, including, for example, gram-positive bacteria, gram-negative bacteria, and fungi. Microorganisms often responsible for healthcare-associated infections and prone to multidrug resistance include, without limitation, methicillin-resistant Staphylococcus aureus (MRSA), Staphylococcus aureus, Enterococcus spp., Enterobacteriaceae (e.g., Escherichia coli, Enterobacter cloacae, Enterobacter aerogenes; Serratia marcesens, Klebsiella pneumoniae, Klebsiella oxytoca, Proteus mirabilis, Proteus vulgaris); Pseudomonas aeruginosa, and Acinetobacter spp., Streptococcus pneumoniae, Staphylococcus epidermidis, Hemophilus influenza; Helicobacter pylori, Salmonella typhimurium, Salmonella typhi, Salmonella paratyphi, E. coli H7:0157, E. coli MG1655, Shigella spp., Neisseria gonorrhoeae, Neisseria meningiditis; anaerobic organisms such as Bacteroides fragilis, Propionibacterium acnes, and Clostridium difficile; organisms of biothreat concern (e.g., Bacillus anthracis; Brucella abortus; Brucella melintensis; Brucella suis; Burkholderia mallei, Burkholderia pseudomallei; Francisella tularensis; Yersinia pestis); mycobacteria such as Mycobacterium tuberculosis complex; M. avium-intracellulare complex; M. kansasii; rapid-growing mycobacteria such as M. fortuitum; M. chelonae; M. abscessus; drug-resistant Candida albicans, Candida auris, and other Candida species; Cryptococcus neoformans and Cryptococcus gattii; unicellular and filamentous fungi such as Acinetobacter baumannii; Issatchenkia orientalis (I. orientalis), Saccharomyces cerevisiae (S. cerevisiae), Candida parapsilosis (C. parapsilosis), Candida neoformans, Candida gattii and Aspergillus fumigatus; and parasites such as Giardia lamblia and Entamoeba histolytica. Other drug-resistant microorganisms include, without limitation, E. coli MDR strains (ATCC, BAA-2340; ATCC, BAA-2469; and ATCC, BAA-2471), Staphylococcus aureus (ATCC, 29213), methicillin-resistant Staphylococcus aureus (ATCC, BAA 1720), Pseudomonas aeruginosa (ATCC, BAA 2113), Klebsiella pneumoniae (ATCC, BAA 2342), vancomycin-resistant Enterococcus faecium (ATCC, 51299), Acinetobacter baumannii (ATCC, BAA 1797) and Enterobacter cloacae (ATCC, BAA 2468).

As used herein, the term “effective amount” refers to those amounts effective to reproducibly reduce the growth of a microorganism, in comparison to their normal levels (i.e., level of growth in the absence of the two-dimensional dispersion of a coated TMC) without undue adverse side effects such as toxicity, irritation, or allergic response. Significant decrease in growth, e.g., as measured using a growth assay, of at least about 30%-40%, and most preferably, by decreases of at least about 50%, with higher values of course being possible. In some cases, the effective amount corresponds to providing coated 2D TMCs at a concentration in the range of about 10 μg ml⁻¹ to about 200 μg ml⁻¹ (e.g., about 10, 12.5, 15, 20, 25, 30, 35, 40, 45, 50, 55, 60, 65, 70, 75, 80, 85, 90, 95, 100, 125, 150, 175, and 200 μg ml⁻¹). In some cases, the effective amount corresponds to a concentration of about 12.5 μg ml⁻¹, 32.5 μg ml⁻¹, 40 μg ml⁻¹, 80 μg ml⁻¹, or 150 μg ml⁻¹. Preferably, two-dimensional dispersions of ssDNA-coated TMCs provided herein exhibit no or minimal toxicity to human cells in vitro, and does not generate acute toxicity in vivo in animal studies. The specific “effective amount” will, obviously, vary with such factors as the particular microorganism, the duration of the treatment, and the specific formulations employed and the structure of the compounds or its derivatives. The optimum effective amounts can be readily determined by one of ordinary skill in the art using routine experimentation. In some cases, the methods of this disclosure comprise coating TMCs in biopolymers known to exhibit some cytotoxicity but, in the materials provided herein, can be used at lower concentrations where they are not cytotoxic.

Unless defined otherwise, all technical and scientific terms used herein have the same meaning as commonly understood by one of ordinary skill in the art to which the invention pertains. All definitions, as defined and used herein, should be understood to control over dictionary definitions, definitions in documents incorporated by reference, and/or ordinary meanings of the defined terms.

The terms “comprising”, “comprises” and “comprised of” as used herein are synonymous with “including”, “includes” or “containing”, “contains”, and are inclusive or open-ended and do not exclude additional, non-recited members, elements, or method steps. The phraseology and terminology used herein is for the purpose of description and should not be regarded as limiting. The use of “including,” “comprising,” “having,” “containing,” “involving,” and variations thereof, is meant to encompass the items listed thereafter and additional items. Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein, the terms “approximately” or “about” in reference to a number are generally taken to include numbers that fall within a range of 5% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

It should also be understood that, unless clearly indicated to the contrary, in any methods claimed herein that include more than one step or act, the order of the steps or acts of the method is not necessarily limited to the order in which the steps or acts of the method are recited.

The present invention has been described in terms of one or more preferred embodiments, and it should be appreciated that many equivalents, alternatives, variations, and modifications, aside from those expressly stated, are possible and within the scope of the invention. The invention will be more fully understood upon consideration of the following non-limiting Examples. The invention will be described in greater detail by way of specific examples. The following examples are offered for illustrative purposes, and are not intended to limit the invention in any manner.

### EXAMPLES

### Example 1—Effects of Two-Dimensional Transition Metal Chalcogenides on Multi-Drug Resistant Organisms

Infections caused by multidrug-resistant (MDR) organisms pose a serious global threat, causing thousands of deaths each year. Ongoing misuse of antibiotic, antifungal, and antiparasitic drugs further add to the growing number of drug-resistant pathogens. Thus, there is a pressing need to develop alternative approaches to combat these pathogenic organisms and terminate their continued spread. The use of engineered nanostructures is currently one of the most promising strategies to overcome microbial drug resistance. Engineered nanostructures provide high surface-to-volume ratios and can exhibit antimicrobial activity as a result of their geometry, composition, or surface functionalization. Two-dimensional ultra-thin nanomaterials can possess excellent antimicrobial properties along with good biocompatibility, which places them at the forefront of nanostructures under investigation for combatting MDR organisms.

Described in this section are studies investigating the abilities of negatively and positively charged biopolymer-wrapped two-dimensional nanosheets of MoSe₂ and other transition metal chalcogenides (TMCs) to kill or inhibit the growth of several MDR organisms. Using single-stranded DNA to encapsulate the TMC nanosheets, we find that MoSe₂ provides the strongest antibacterial activity against Escherichia coli from a set of eight different TMCs analyzed and that larger MoSe₂ sheets exhibit higher activity than smaller ones. MoSe₂ formulations that coat the nanosheets using the cationic antimicrobial biopolymers poly-L-lysine and chitosan enable complete eradication of MDR bacterial strains and fungi, respectively. Electron microscopy studies and biochemical assays reveal that the remarkable antimicrobial activity of the MoSe₂ formulations is due to a multimodal mechanism of action. The MoSe₂ nanosheets exploit electrostatic interactions to promote interaction with the cell wall, efficiently puncture the cell wall using their atomically thin hydrophobic structure, and interfere with cellular metabolism by inducing oxidative stress.

Results and Discussion:

Preparation of 2D TMCs as Nanoscale Antimicrobials

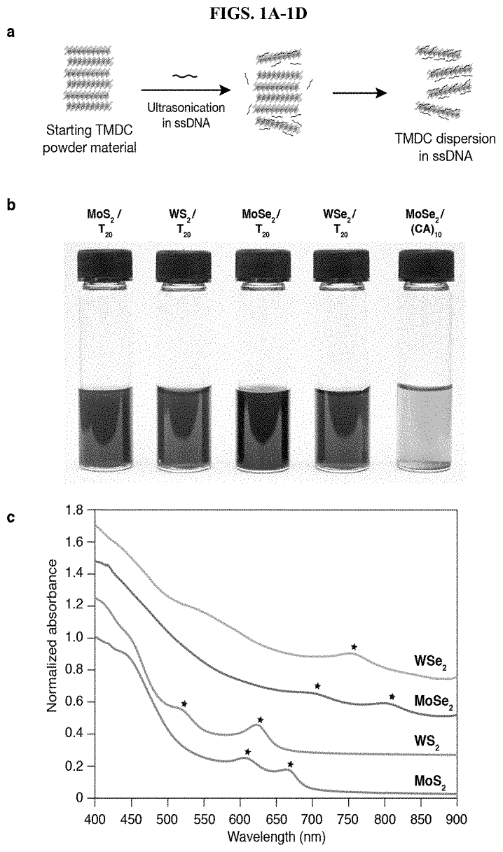

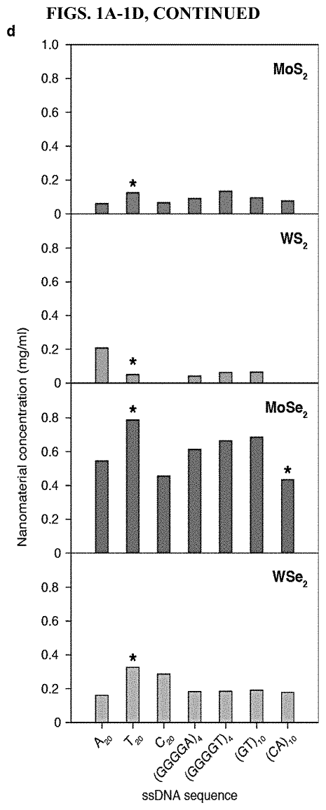

Many recent strategies for exfoliation have been based on the ultrasonication of bulk TMC powders in solution phase through energetic agitation. The waves propagate through the solvent causing alternating high and low-pressure cycles that aid exfoliation. These cycles generate two different forms of energy inputs; namely, vibration and cavitation forces. The process for liquid exfoliation relies on the continuous exposure of the bulk TMC crystals to these energetic forces to continually thin them. With enough ultrasonic power, vibrational forces can overcome the van der Waals forces within TMC flakes, enabling exfoliation of TMCs and other compounds down to individual layers. Following exfoliation of 2D nanosheets, amphiphilic moieties containing a hydrophilic and a hydrophobic group are used to stabilize the dispersions of intrinsically hydrophobic TMCs in aqueous solution. The initial dispersion is centrifuged to remove poorly dispersed and dense particles, and the supernatant harvested to use for further studies. Liquid-mediated exfoliation processes are a practical and low-cost approach to large-scale nanosheet production since processing volumes can be scaled-up substantially and make use of commercially available large-volume sonication and centrifugation equipment. Inspired by previous work on the use of surfactants and DNA for wrapping single-walled carbon nanotubes, studies were conducted to disperse TMCs (e.g., MoSe₂, MoS₂, WSe₂, WS₂) in a variety of different ssDNA sequences. Based on the amphiphilic structure of ssDNA, we anticipated that the hydrophobic DNA bases would undergo strong π-stacking interactions with the hydrophobic surfaces of the TMDCs, enabling the hydrophilic sugar-phosphate ssDNA backbone to interface with surrounding water molecules. The negatively charged phosphate groups in the backbone would also provide strong electrostatic repulsion to suspend each flake in solution and prevent nanosheet restacking (see FIG. 1A). Given the sequence dependence of ssDNA dispersions of carbon nanotubes(Zheng et al., 2003), the ability of seven 20-nucleotide (nt) ssDNAs containing different sequences of the four canonical DNA bases adenine (A), cytosine (C), guanine (G), and thymine (T) to disperse the TMDCs was investigated. These sequences contained 20-mer repeats of the bases (A₂₀ (SEQ ID NO: 4), C₂₀ (SEQ ID NO: 5), and T₂₀ (SEQ ID NO: 2)); or they consisted of repeated pairs of non-complementary bases ((GT)₁₀ (SEQ ID NO: 1), (CA)₁₀ (SEQ ID NO: 3)). Since long guanine repeats are challenging to synthesize, the guanine-rich ssDNAs (GGGGA)₄ (SEQ ID NO: 6) and (GGGGT)₄ (SEQ ID NO: 7) were studied.

Bulk TMDC powders for TMCs MoSe₂, MoS₂, WSe₂, WS₂, Bi₂Se₃, Bi₂S₃, SnSe, and SnS were probe ultrasonicated in aqueous solutions containing 0.2 mg mL⁻¹ of synthetic ssDNA. The ultrasonicated solutions were then centrifuged and the resulting supernatants harvested. FIG. 1B provides a photograph of the strongly colored and stable 2D TMDCs dispersions prepared using the common sequence T₂₀ (SEQ ID NO: 2), which was an effective sequence for dispersing all four of the TMDCs. Optical absorbance spectroscopy of the dispersions confirmed successful exfoliation of the TMDCs revealing the characteristic excitonic transition peaks for the four compounds (FIG. 1C). The resulting TMC/ssDNA nanosheets were then tested in antimicrobial assays as described below.

The sonication technique was also used to prepare dispersions of MoSe₂ coated in a mixture of poly-L-lysine (PLL) and the Pluronic F77 block copolymer. The resulting MoSe₂/PLL/F77 were prepared in a two-step procedure where bulk MoSe₂ was first sonicated in the presence of PLL and then further stabilized by sonicating in Pluronic F77 (see Materials and Methods). Pluronic F77 is a biocompatible non-ionic difunctional block copolymer surfactant terminating in primary hydroxyl groups. The long alkyl chain of Pluronic F77 keeps the nanosheets from reaggregating and the positively charged amine (—NH₂) groups on the PLL act as a hydrophilic group exposed towards the aqueous medium. As a result of this mixed surface functionalization, the MoSe₂/PLL/F77 nanosheets have a net positive charge, in contrast to the negative charge of the TMC/(GT)₁₀ (SEQ ID NO: 1) nanosheets. It is important to note that Pluronic F77 constitutes just one member from the diverse family of Pluronic amphiphilic, non-ionic block copolymers. These biocompatible polymers are available in a wide range of molecular weights with different degrees of hydrophilic and hydrophobic character depending on the sizes of the constituent polymer blocks. In addition, the closely related family of Tetronic copolymers can also be used for stabilization. It is possible that, in some cases, other Pluronics or a Tetronic copolymer may provide better performance than Pluronic F77 when integrated into the MoSe₂/PLL complexes.

A second cationic preparation of MoSe₂ nanosheets was made using the antimicrobial biopolymer chitosan. MoS₂/chitosan dispersions were made by ultrasonicating the bulk powders in 1% chitosan. Chitosan is a biocompatible cationic linear polysaccharide composed of randomly distributed β-(1-4)-linked D-glucosamine and N-acetyl-D-glucosamine. The polysaccharide chain acts as a bulky group keeping the nanosheets from re-aggregating due to steric repulsion and the cationic amine (—NH₂) groups of D-glucosamine acts as a hydrophilic outer layer.

The concentrations of the TMDCs dispersed in the synthetic ssDNAs were determined using inductively coupled plasma mass spectrometry (ICP-MS). MoSe₂ and WSe₂ yielded the highest concentration dispersions with loadings above 250 μg mL⁻¹ obtained for optimal ssDNA sequences. The sulfur-containing MoS₂ and WS₂ displayed lower loadings, in general, with typical concentrations of 50 μg mL⁻¹. The stability of DNA-wrapped TMDCs was tested using zeta potential, with MoSe₂ and WSe₂ being the most stable with a zeta potential of −42.13 and −40.13 mV, whereas MoS₂ and WS₂ being the least stable one with zeta potential of −21.13 and −20.07. Significant variations in the concentration of dispersed TMDCs depending on the sequence of ssDNA used and the composition of the TMDC were observed. In general, the highest concentration dispersions were obtained for the T₂₀ (SEQ ID NO: 2) sequence whereas (CA)₁₀ (SEQ ID NO: 3) was the weakest out of the sequences studied. The study also revealed thymine and guanine showed better affinity towards all TMDCs compared to cytosine and adenosine. The DNA coverage of MoSe₂/T₂₀ (SEQ ID NO: 2) was further evaluated using thermogravimetric analysis, demonstrating ˜8% DNA coverage on the surface of TMDCs. The repeating dinucleotide sequence GT was also tested with ssDNAs of different lengths, in particular 10-nt, 20-nt, and 40-nt lengths. We found that (GT)₁₀ (SEQ ID NO: 1) provided the highest concentrations among the three, and that MoS₂ ssDNA with (GT)₅ (SEQ ID NO: 8) was unstable and aggregated within a few hours.

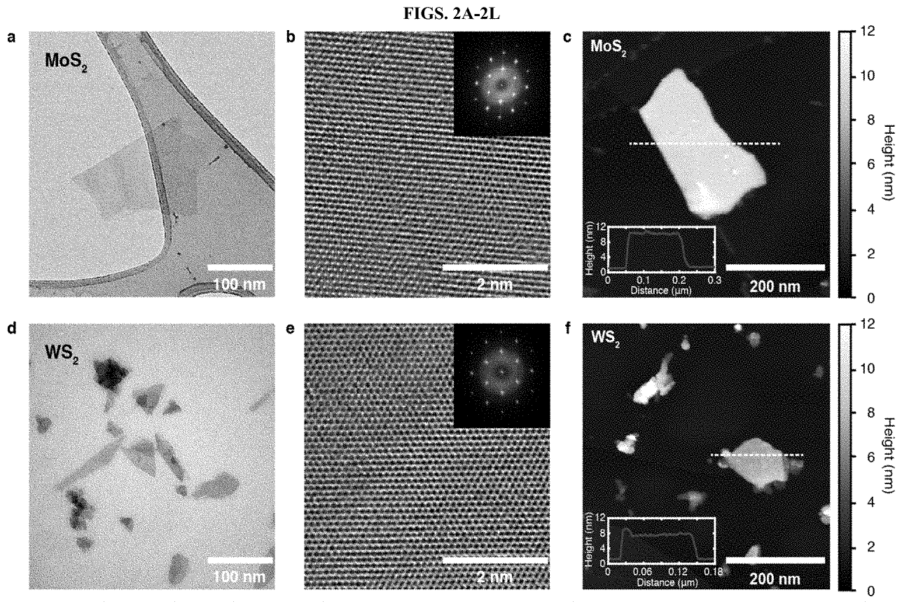

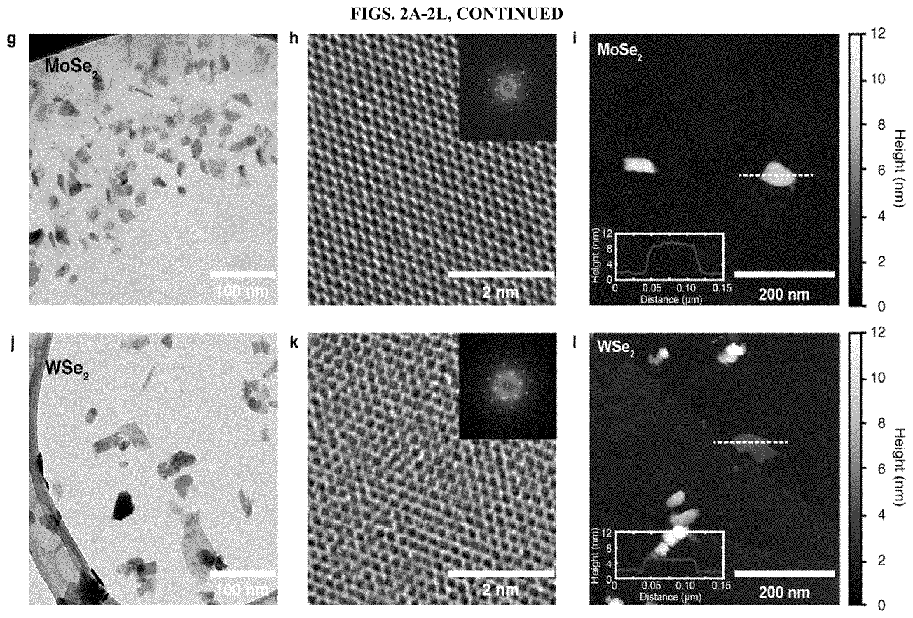

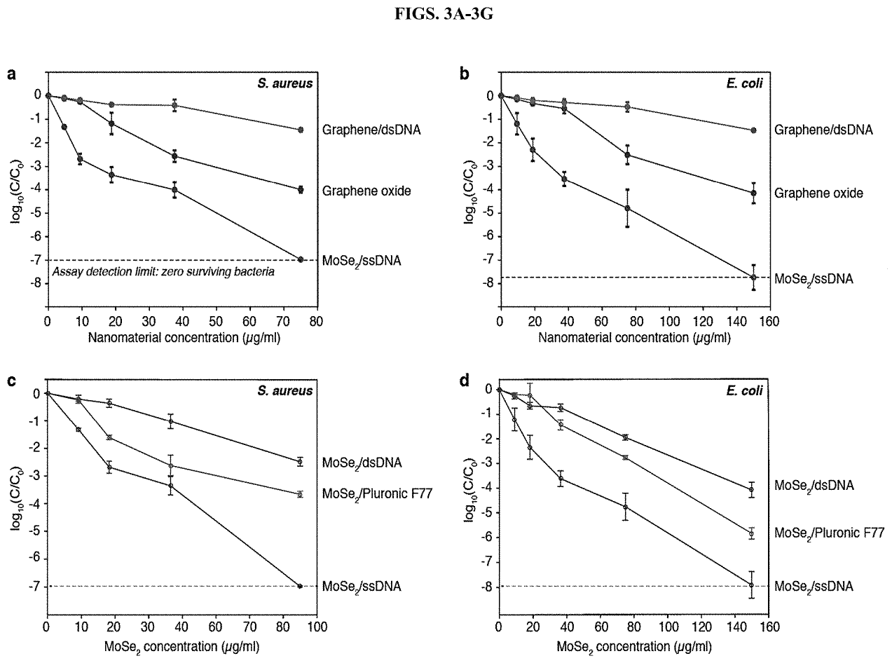

The successful exfoliation of 2D TMDCs using low molecular weight ssDNA was further characterized using low and high-resolution transmission electron microscopy (TEM and HRTEM) and atomic force microscopy (AFM) as shown in FIGS. 2A-2L. TEM imaging of the exfoliated TMDCs revealed their geometries as 2D nanosheets. The average lateral size of nanoflakes decreased in order from MoS₂, WS₂, MoSe₂, and WSe₂ (FIGS. 2a, 2d, 2g, and 2j). MoS₂ yielded nanoflakes with lateral dimensions of approximately 80-100 nm, WS₂ and MoSe₂ from 60-70 nm, and WSe₂ less than 50 nm. AFM images of MoS₂ confirmed the presence of flakes with extended lateral dimensions with thicknesses of around 10 nm. The WS₂ dispersion showed the second largest flakes with thicknesses less than 8 nm, whereas MoSe₂ and WSe₂ exhibited the smallest flakes with thicknesses around 8 nm and 4 nm. The measured thicknesses of TMDC flakes also include a uniform layer of ssDNA, which covers both sides of the TMDC flakes. The AFM and TEM data obtained correlate well with each other and both showed successful ssDNA-based exfoliation of TMDCs to produce few-layer TMDCs. We further studied the crystal structure of DNA-dispersed TMDCs using HRTEM. These images show that the TMDCs retain the expected hexagonal lattice structure of pristine 2H phase of the TMDCs following encapsulation by short ssDNAs.

Antibacterial Activity of MoSe₂/ssDNA

Since MoSe₂ dispersions provided the highest concentrations of 2D TMDCs overall and were coated with a biocompatible polymer, the effect of MoSe₂/T₂₀ (SEQ ID NO: 2) dispersions on human and bacterial cells was studied. To assess their toxicity, MoSe₂/T₂₀ (SEQ ID NO: 2) nanosheets were first added to cultures of the model HeLa human epithelial carcinoma cell line. Concentrations of MoSe₂/T₂₀ (SEQ ID NO: 2) ranging from 25 μg mL⁻¹ to 250 μg mL⁻¹ were applied to cultures for 24 hours and cell viability was determined using the colorimetric MTT assay. No significant toxicity was observed for MoSe₂/T₂₀ (SEQ ID NO: 2), with 100% cell viability at all concentrations tested.

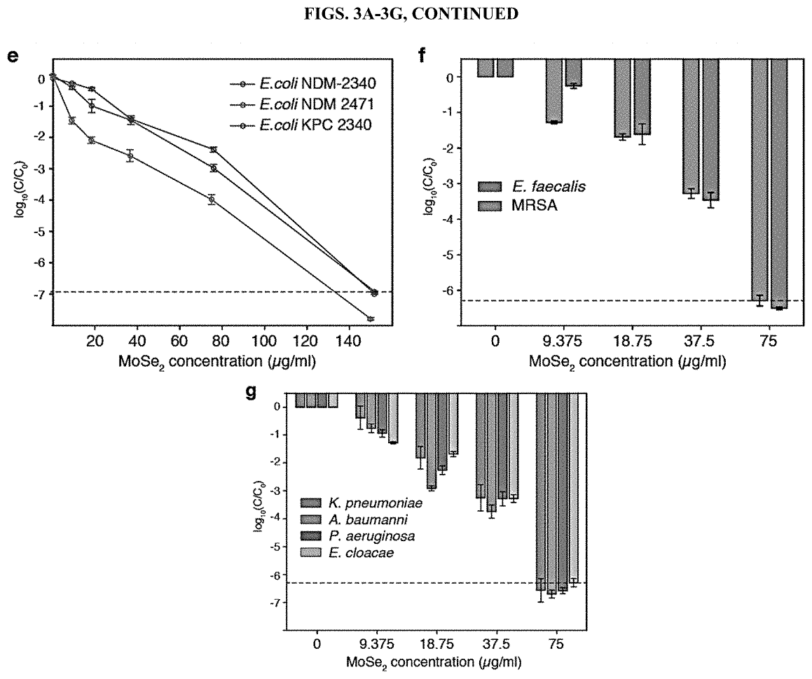

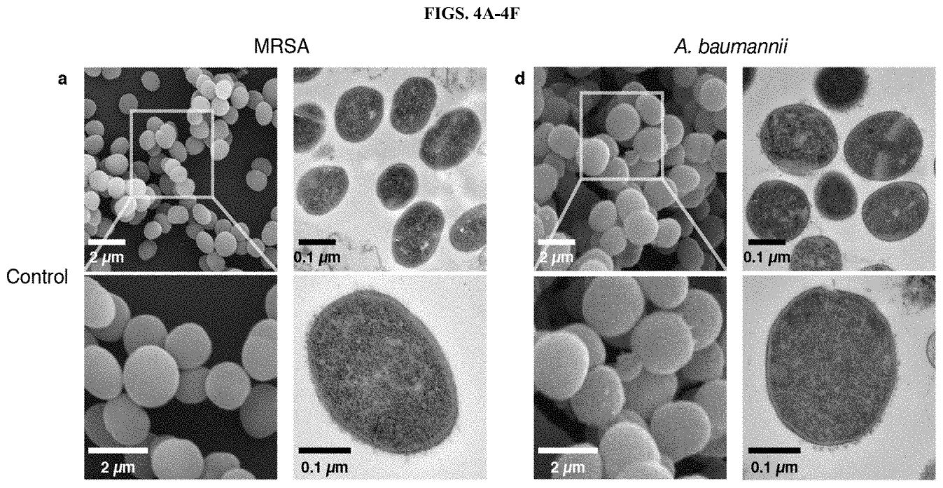

Having confirmed the biocompatibility of MoSe₂/T₂₀ (SEQ ID NO: 2) nanosheets, we next evaluated their effect on two representative strains of gram-negative and gram-positive bacteria, E. coli MG1655 and Staphylococcus aureus subsp. aureus (ATCC 29213), respectively. For determination of antibacterial activity, logarithmic-phase bacteria at a concentration of 10⁷ cell-forming units per mL (CFU mL⁻¹) were treated with MoSe₂/T₂₀ (SEQ ID NO: 2) dispersions at different concentrations for 4 hours. Microdilutions of the treated bacteria were then applied to agar and incubated overnight for colony counting the next day. The MoSe₂/T₂₀ (SEQ ID NO: 2) dispersions displayed exceptional antibacterial activity against both species of bacteria. We observed 100% elimination of E. coli at a concentration of 150 μg mL⁻¹ MoSe₂/T₂₀ (SEQ ID NO: 2) (FIG. 3A). For S. aureus, MoSe₂/T₂₀ (SEQ ID NO: 2) displayed more potent activity, completely eliminating the gram-positive strain at a concentration of only 75 μg mL⁻¹ (FIG. 3C).

Recently, the antibacterial activity of carbon-based 2D materials has drawn wide attention. In a recent report, Liu et al. carried out a comparative study of antibacterial activities of four different graphene-based materials (graphite, graphite oxide, graphene oxide and reduced graphene oxide), with graphene oxide (GO) showing the strongest bactericidal effect. Thus, we compared the antibacterial activity GO with respect to MoSe₂/T₂₀ (SEQ ID NO: 2) on wild-type E. coli MG1655 strain under the same experimental conditions. GO and MoSe₂/T₂₀ (SEQ ID NO: 2) were incubated with E. coli MG1655 for 4 hours, plated on agar, and cell viability was determined by colony counting. At 150 μg ml⁻¹, MoSe₂/T₂₀ (SEQ ID NO: 2) showed 3 orders of magnitude higher antibacterial killing as compared to graphene oxide (FIG. 3B). We further compared the antibacterial activity of MoSe₂/T₂₀ (SEQ ID NO: 2) and GO over different concentrations and found that MoSe₂/T₂₀ (SEQ ID NO: 2) showed stronger bactericidal properties than GO, with over an order of magnitude higher potency at 80 and 100 μg mL⁻¹ concentrations. We further compared the antibacterial activity of GO with respect to MoSe₂/T₂₀ (SEQ ID NO: 2) on gram-positive S. aureus strain under the same experimental conditions. At 150 μg ml⁻¹, MoSe₂/T₂₀ (SEQ ID NO: 2) showed 3 orders of magnitude higher antibacterial activity compared to GO. Also, antibacterial efficiency of MoSe₂/T₂₀ (SEQ ID NO: 2) is more than 1 order of magnitude higher at all the tested concentrations.

To determine if the short synthetic DNA coating contributed to the enhanced antibacterial activity of the MoSe₂ nanosheets, we then carried out comparative studies with MoSe₂ encapsulated by three biocompatible polymers: T₂₀ (SEQ ID NO: 2) ssDNA, long genomic double-stranded DNA (dsDNA) purified from salmon testes, and the block copolymer Pluronic F77. A recent report by Bang et al. demonstrated that WSe₂ suspended using genomic dsDNA had two-fold higher antibacterial activity than GO. Pluronic F77 has been widely used for preparing biocompatible dispersions of 2D nanosheets and consists of a hydrophobic polypropylene oxide block flanked by two hydrophilic polyethylene oxide regions. In our experiments, we found that MoSe₂/T₂₀ (SEQ ID NO: 2) showed significantly stronger bactericidal properties compared to MoSe₂/dsDNA and MoSe₂/F77 for nearly all the concentrations tested and provided stronger enhancements as the concentration increased (FIG. 3D). At 150 μg mL¹ concentrations, MoSe₂/T₂₀ (SEQ ID NO: 2) completely eradicated E. coli, which corresponded to the elimination of all 10⁷ cells treated, while the activity of MoSe₂/dsDNA and MoSe₂/F77 was 2.2 and 3.2 orders of magnitude weaker, respectively. Also, MoSe₂/T₂₀ (SEQ ID NO: 2) showed 4 and 3 orders of magnitude higher killing towards S. aureus compared to MoSe₂/long genomic DNA and MoSe₂/F77. The variation in antibacterial properties with different encapsulating polymers is attributed to changes in the thickness of the polymer coating each MoSe₂ nanosheet. The Pluronic F77 employs bulky polyethylene oxide blocks to suspend the MoSe₂ in the aqueous environment; however, these polymer blocks discourage strong interactions with the bacterial cell wall. For the genomic dsDNA, the use of long DNA sequences prevents the formation of a conformal ssDNA coating around the MoSe₂ and thereby increases the effective thickness of the nanosheets. In contrast, the short length of the T₂₀ (SEQ ID NO: 2) sequence enables the ssDNA to effectively coat and spread out along the MoSe₂ surface and yields an ultrathin material to promote piercing of the bacterial cell wall.

Antibacterial Studies Against MDR Bacteria

The recent emergence of nosocomial infections by bacteria with broad-spectrum resistance to antibiotics in hospitals and clinics requires novel antibacterial strategies. Assays were designed to determine if the MoSe₂/T₂₀ (SEQ ID NO: 2) dispersions retained their exceptional bactericidal activity against several multidrug resistant strains. First evaluated were three strains of multi-drug resistant (MDR) E. coli carrying resistance to carbapenems, a class of antibiotic agents often used to combat drug-resistant infections. E. coli NDM 2469 and E. coli NDM 2471 strains both carry the New Delhi metallo-β-lactamase (NDM), a recently identified carbapanemase, while E. coli KPC 2340 carries the Klebsiella pneumoniae carbapenemase (KPC). All three strains exhibit broad-spectrum resistance to multiple families of antibiotics. Antibiotic resistance tests conducted by ATCC on the NDM 2469 and NDM 2471 strains indicated that these strains were resistant to 33 and 32 out of 35 antibiotics tested, respectively. Studies by ATCC on E. coli KPC 2340 indicated that it was resistant to 30 out of the 34 antibiotics evaluated. The three MDR E. coli strains were exposed to different concentrations of MoSe₂/T₂₀ (SEQ ID NO: 2) over 4 hours and surviving cells counted after plating microdiluted samples. No viable E. coli bacteria were observed after treatment with 150 μg ml⁻¹ of MoSe₂/T₂₀, (SEQ ID NO: 2) and the killing efficiency increased with increasing concentration (FIG. 3E). Furthermore, at lower concentrations of MoSe₂/T₂₀ (SEQ ID NO: 2), higher killing efficiency was observed for NDM E. coli strains as compared to KPC E. coli strains, as shown in FIG. 3E. At 100 μg ml⁻¹, MoSe₂/T₂₀ (SEQ ID NO: 2) showed 99.98% and 99.8% cell killing for NDM strains, whereas 99.5% cell killing was observed for KPC strains. A representative gram-positive MDR strain of methicillin-resistant S. aureus (MRSA) was also exposed to MoSe₂/T₂₀ (SEQ ID NO: 2). The MoSe₂ nanosheets provided more efficient bactericidal activity against the S. aureus strain, completely eliminating the bacteria at a concentration of 75 μg mL⁻¹.

After successful eradication of MDR E. coli, we further evaluated antibacterial efficiency of MoSe₂/T₂₀ (SEQ ID NO: 2) on nosocomial MDR clinical isolates that are members of the ‘ESKAPE’ group of pathogens. First evaluated was the efficiency of MoSe₂/T₂₀ (SEQ ID NO: 2) against two well-known gram-positive clinical strains, methicillin-resistant Staphylococcus aureus (MRSA) and vancomycin-resistant Enterococcus faecium (VRE). MRSA, which is responsible for 25 to 50% of nosocomial infections, expresses the mecA gene that provides it with resistance to a broad spectrum of β-lactam antibiotics. VRE, which causes urinary tract infections, is resistant to all β-lactam antibiotics and last resort antibiotics for treating vancomycin. After 4 hours of treatment, 75 μg ml⁻¹ MoSe₂/T₂₀ (SEQ ID NO: 2) successfully eliminated 10⁷ orders of VRE and MRSA (FIG. 3F). The efficiency of MoSe₂/T₂₀ (SEQ ID NO: 2) against the following gram-negative ‘ESKAPE’ strains was examined: Klebsiella pneumoniae, Acinetobacter baumannii, Pseudomonas aeruginosa, and Enterobacter. Antibiotic resistance conducted by ATCC on Pseudomonas aeruginosa and Acinetobacter baumanii strains, demonstrated resistance to 15 and 31 different classes of antibiotics, whereas Enterobacter and Klebsiella pneumoniae demonstrated resistance to 36 different classes of antibiotics. After 4 hours of treatment, 75 μg ml⁻¹ of MoSe₂/T₂₀ (SEQ ID NO: 2) successfully eradicated all the four gram-negative MDR strains (FIG. 3G). The successful eradication of these “Superbugs” demonstrates that MoSe₂/T₂₀ (SEQ ID NO: 2) provide an effective strategy for combating these drug resistant bacteria.

Multimodal Antibacterial Mechanism of MoSe₂/T₂₀

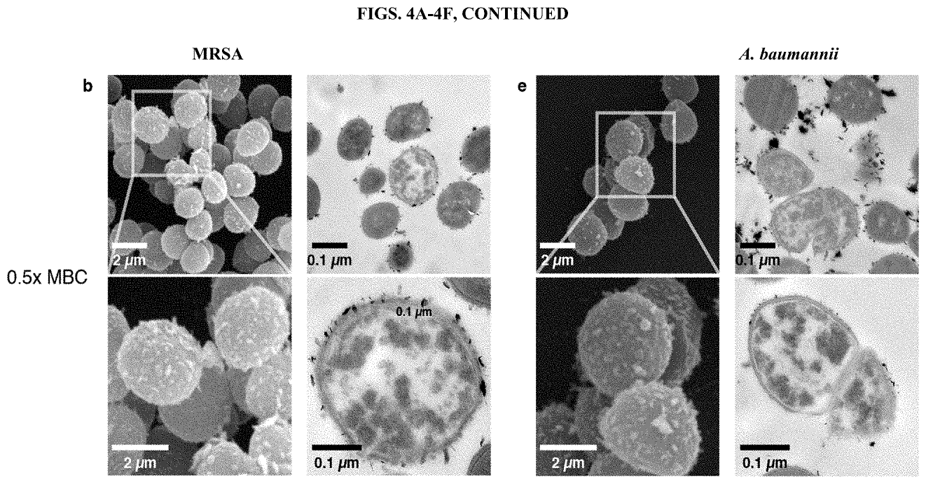

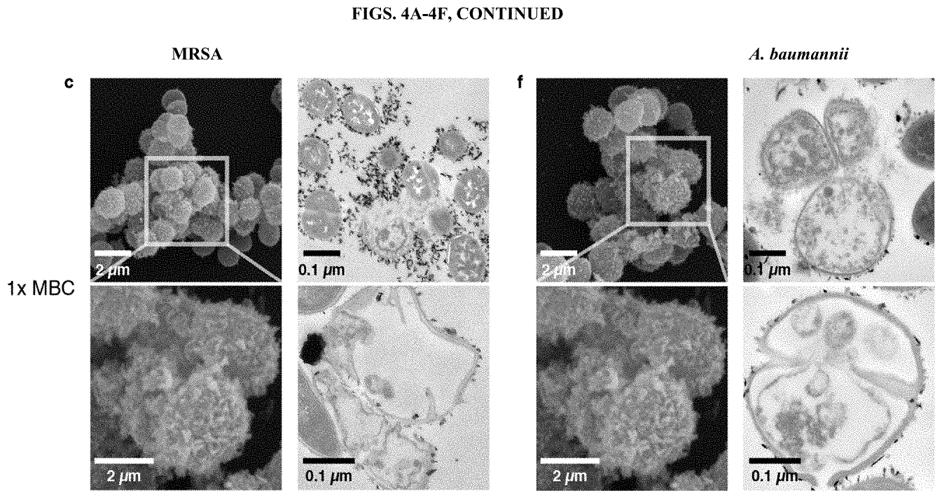

We carried out SEM and TEM studies to investigate the effect of MoSe₂/T₂₀ (SEQ ID NO: 2) interactions on the cell morphology of MRSA and A. baumannii cells. SEM images of untreated MRSA and A. baumannii samples showed no significant morphological changes (FIGS. 4A and 4D), and TEM images of untreated MRSA and A. baumannii showed an intact cytoplasm (FIGS. 4A-and 4D). SEM images of MRSA and A. baumannii treated at 0.5×MBC (37.5 μg ml-1) of MoSe₂/T₂₀ (SEQ ID NO: 2) showed formation of small blob-like structures (FIG. 4B), which further increased at 1×MBC (75 μg/ml) (FIG. 4C) The formation of small blobs can be attributed to the fact the interactions of nanomaterials with their sharp edges, causes breakdown of the cell membrane. TEM images of MRSA and A. baumannii showed TMDC flakes encapsulating and penetrating the cell membranes of bacteria (FIGS. 4B, 4E and 4C, 4F). In both gram-negative A. baumannii and gram-positive MRSA, TEM images show the presence of void spaces in the cytoplasm at 0.5×MBC, which further increased with increasing nanomaterial concentrations. The formation of small grooves structure in the cellular membrane, leads to disturbance in the cytoplasm and finally leakage of the cytoplasm from the cells. The nature of damage to the cellular membrane and cytoplasm is identical for both gram-positive and gram-negative bacteria, which further supports the broad-spectrum antibacterial nature of nanomaterials.

The microscopic analysis of TMDC-treated bacteria indicate that the TMDC flakes caused severe damage to the peptidoglycan of the plasma membranes. The damaged membrane in turn fails to hold the turgor pressure of the cytoplasm and leads to leakage of the cytoplasm. The observed effect is consistent with other reports of the antibacterial mechanism of carbon-based nanomaterials, which act as “nanoknives” that can interfere with membrane integrity as a result of their atomically sharp graphene edges (Hu et al., 2010; Tu et al., 2013; Zhang et al., 2016). The theoretical and experimental studies of antibacterial mechanisms of graphene-based nanomaterials have shown that they act like blades penetrating through bacterial cell membranes, causing physical damage, and leading to leakage of cytoplasm. Based on previous studies, we hypothesize that the presence of DNA on TMDC surfaces increases their hydrophilicity, which encourages them to make contact with the outer surfaces of the bacteria. Once in close proximity, the sharp edges of the MoSe₂/T₂₀ (SEQ ID NO: 2), which are more readily exposed as a result of the conformal ssDNA coating, are better able to interfere with the membrane to trigger cell death.

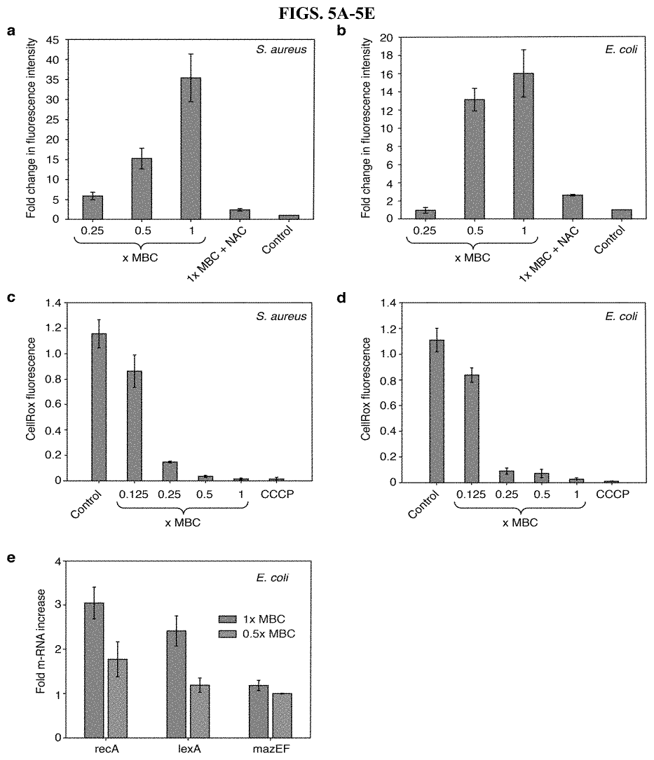

We further evaluated the antibacterial killing mechanism MoSe₂/T₂₀ (SEQ ID NO: 2) using the commonly used probe 3,3′-Diethyloxacarbocyanine Iodide (DiOC₂). DiOC₂ is a cyanine dye, which permeates the cell membrane exhibiting green fluorescence, and undergoes aggregation in polarized cells leading to fluorescence shifting from green to red. We evaluated the change in membrane potential after nanomaterial interactions on two different strains of bacteria, gram-positive S. aureus and gram-negative E. coli. For the membrane potential assay, we treated the bacteria at four different concentrations, 1×MBC, 0.5×MBC, 0.25×MBC, and 0.125×MBC. The carbonyl cyanide 3-chlorophenylhydrazone, a known membrane ionophore, was used as a positive control for the membrane potential assay. With increasing nanomaterial concentrations, the fluorescence shifts towards green channel and leads to a lowering of the red/green fluorescence intensity ratio. The change in fluorescence intensity ratio indicates that interactions with the nanomaterial triggers depolarization of the cell. After determining membrane potential, we further studied the induction of oxidative stress using fluorogenic probe CellROX. The oxidative stress induced after interaction with the nanomaterial was studied for both gram-positive S. aureus and gram-negative E. coli at three different concentrations: 1×MBC, 0.5×MBC, and 0.25×MBC. At 1×MBC, MoSe₂/T₂₀ (SEQ ID NO: 2) induces 42-fold and 16-fold higher oxidative stress for S. aureus and E. coli, respectively, compared to untreated samples (FIGS. 5C and 5D). Furthermore, oxidative stress in the cell rises with increasing nanomaterial concentration. The addition of antioxidant N-acetylcysteine at 1×MBC of nanomaterial concentration reduces the oxidative stress by 19-fold and 6-fold in S. aureus and E. coli, respectively, which further demonstrates the generation of cellular oxidative stress after interactions with nanomaterials.

After determination of membrane potential and oxidative stress generation in bacterial cells, we further evaluated the impact of this stress on DNA damage. Damage to DNA generally triggers programmed cell death. There are two well-known pathways for programmed cell death, RecA and mazEF pathways. The mazEF pathways refers to toxin-antitoxin module of bacteria. mazF genes generally encode for a toxic endoribonuclease protein MazF, which quickly degrades mRNA whereas mazE genes lead to secretion of the antitoxin mazE, which neutralizes the effect of MazF. Under stressful conditions, MazE is quickly degraded by ClpPA serine protease, which leads to the presence of higher toxic protein and in turn cell death. The SOS response pathway refers to inducible pathways that are responsible for DNA repair. There are two key proteins which is responsible for this SOS response pathways, repressor LexA and inducer RecA. In absence of DNA damage, LexA dimer binds to palindromic sequence of the DNA SOS box, inhibiting expression of RecA genes. Under DNA damage, RecA pathway is activated leading to self-cleavage of LexA and activation of RecA pathways, which leads to cell death. We studied the programmed cell death pathways of E. coli after interaction with 0.5×MBC and 1×MBC of MoSe₂/T₂₀ (SEQ ID NO: 2). After interactions of nanomaterials with E. coli, 0.5×MBC and 1×MBC showed 2-fold and 3-fold increase in mRNA expression of RecA levels, whereas mazEF level is essentially unchanged for both the tested concentrations. These studies clearly demonstrate involvement of two pathways towards the cell death after interaction with MoSe₂/T₂₀ (SEQ ID NO: 2).

The antibacterial activity of MoSe₂-ssDNA can be summarized as a three-step process. First, the sharp edge of nanomaterials undergo insertion into bacterial cell membrane, causing ruptures. These ruptures lead to depolarization of the cell. The alteration of membrane potential, in turn induces oxidative stress, causing damage to the cellular machinery and in turn cell death.

MoSe₂/ssDNA showed stronger bactericidal property towards gram-positive strains than gram-negative strains at all the concentrations we tested. This trend correlates with previously obtained antibacterial activity for carbon-based nanomaterials. The difference in killing efficiency between gram-negative and gram-positive bacterial strains can be explained by their different outer structures. Gram-negative bacteria have outer membranes, which are made up of a lipid bilayer and thin peptidoglycan layer, whereas gram-positive bacteria are made up of a thick peptidoglycan layer and no outer membrane. The outer membrane of gram negative bacteria is made up of negatively charged glucosamine disaccharides called lipopolysaccharides, whereas gram positive bacteria contain thicker peptidoglycan layer with long anionic polymer called teichoic acids threading out from the peptidoglycan layer. The presence of more negatively charged surfaces on gram-negative bacteria provides higher resistance to negatively charged nanomaterials. Furthermore, the absence of outer membrane on gram-positive bacteria makes it more susceptible to membrane damage by direct interaction with the sharp edges of nanomaterial flakes. The absence of LPS coupled with lack of outer membrane on the surfaces of gram-positive bacteria makes them more susceptible to membrane damage by sharp edges of negatively charged nanomaterials.

Conclusion:

Using short synthetic ssDNA sequences, these assays demonstrate successful preparation of stable dispersions of 2D TMDCs in aqueous solutions and demonstrated their remarkable antibacterial performance. Exfoliation of few-layer TMDC nanosheets was confirmed using optical absorbance and TEM and AFM imaging. Concentration measurements using ICP-MS identified MoSe₂ as the TMDC most effectively dispersed using synthetic ssDNA. Owing to the biocompatibility of the ssDNA coating, we studied the effect of the MoSe₂ nanosheets encapsulated by T₂₀ (SEQ ID NO: 2) ssDNA against human and bacterial cells. While no toxicity was observed against a human cell line, we observed potent bactericidal activity for the MoSe₂/T₂₀ (SEQ ID NO: 2) nanosheets against multiple strains of gram-negative and gram-positive bacteria. In side by side comparisons, the bactericidal efficiency of MoSe₂/T₂₀ (SEQ ID NO: 2) nanosheets exceeded that of the most widely studied two-dimensional antibacterial material graphene oxide by more than 1000-fold. Furthermore, it exceeded the reported activity of Ti₃C₂Tₓ, a recently developed two-dimensional antibacterial, by more than 100-fold and 10-fold against gram-positive and gram-negative bacteria, respectively. MoSe₂ prepared using genomic DNA or with a Pluronic F77 coating alone were much less effective at eliminating the bacteria, demonstrating the critical importance of an optimized, conformal ssDNA coating for enhanced antibacterial activity. Studies of both E. coli and MRSA cells treated with the MoSe₂/T₂₀ (SEQ ID NO: 2) revealed that the nanosheets aggressively interact with the cell walls of the bacteria, acting as nanoknives that create holes and folds in the membrane to induce cell death. Furthermore, MoSe₂/T₂₀ (SEQ ID NO: 2) successfully eradicated clinical isolates of the ‘ESKAPE’ strains, demonstrating its potential as a broad-spectrum antibacterial material against ‘Superbugs’. These results are consistent with literature understanding of DNA adsorption on TMDC surfaces and sequence-dependent molecular dynamics simulations.

These results not only demonstrate that 2D MoSe₂/ssDNA can exhibit antibiotic-like activity against multidrug resistant bacteria, but also emphasize the importance of employing optimized encapsulation agents to enhance the effectiveness of two-dimensional nanoknives. In particular, the use of a conformal ssDNA coating suggests that an array of other biocompatible biomolecules can be interfaced with intrinsically antimicrobial 2D nanomaterials to generate effective new tools to combat the growing threat of antimicrobial drug resistance.

Material and Methods:

Solution Phase Dispersion of TMDCs in ssDNA

In a typical experiment, TMDC powder was added to a 5-mL aqueous solution containing 1 mg of single-stranded DNA (Integrated DNA Technologies). MoSe₂ and WSe₂ bulk powder (Sigma Aldrich) at a mass of 200 mg was used for each dispersion, while a lower mass of 100 mg of MoS₂ and WS₂ (Sigma Aldrich) led to higher concentration dispersions for these compounds. The resulting mixture was ultrasonicated with a 13-mm tip f at a power level of 12 W for 2 hours (Branson Digital Sonifier 450D). After ultrasonication, the sample was centrifuged at 5,000 g for 5 minutes followed by 21,000 g for 1 minute to remove the unexfoliated material. The supernatant of ssDNA-encapsulated TMDC nanosheets was then carefully decanted for study.

TEM Analysis of ssDNA-Dispersed TMDC Nanosheets

The sample was prepared using drop casting method. Briefly, 6 μl of dispersed solution was drop casted onto holey carbon copper grids and imaged using a Phillips CM-12 TEM.

AFM Analysis of ssDNA-Dispersed TMDC Nanosheets

Highly oriented pyrolytic graphite (HOPG) substrates (SPI Inc.) were freshly cleaved using adhesive tape. Then the dispersions of nanomaterials were spin-coated onto the substrates at 2500 rpm. The spin coated samples were annealed in vacuum with 200 sccm of ultrahigh pure argon gas flow at 300° C. for 3 hours to remove organic residues. AFM images were taken using a Multimode V system (Bruker Inc.) in ScanAsyst mode with ScanAsyst-Air tips and image processing was conducted using Gwyddion.

Antibacterial Studies.

The antibacterial activity of MoSe₂-ssDNA was studied using wild type E. coli strain MG1655 (ATCC, 700926), three E. coli MDR strains (ATCC, BAA-2340; ATCC, BAA-2469; and ATCC, BAA-2471), Staphylococcus aureus (ATCC, 29213), methicillin-resistant Staphylococcus aureus (ATCC, BAA 1720), Pseudomonas aeruginosa (ATCC, BAA 2113), Klebsiella pneumoniae (ATCC, BAA 2342), vancomycin-resistant Enterococcus faecium (ATCC, 51299), Acinetobacter baumannii (ATCC, BAA 1797) and Enterobacter cloacae (ATCC, BAA 2468). LB medium (Sigma Aldrich) and LB agar (Sigma Aldrich) were used to grow E. coli strain MG1655. TSB broth (Sigma Aldrich) and TSB agar (Sigma Aldrich) were used to grow Staphylococcus aureus, methicillin-resistant Staphylococcus aureus, Pseudomonas aeruginosa and Acinetobacter baumannii whereas vancomycin-resistant Enterococcus faecium were grown in BHB broth (Sigma Aldrich) and BHB agar (Sigma Aldrich) in presence of 4 mcg/ml of vancomycin. MHB broth (Sigma Aldrich) and MHB agar (Sigma Aldrich) was used to grow Klebsiella pneumoniae. Single colonies were picked from agar plates and allowed to grow overnight in 5 ml of culture medium. Then, the sample was diluted 100 times in medium and allowed to grow until it reached 0.3 OD. Cultures were centrifuged at 2000 rpm for 10 minutes, and pellets were washed three times in phosphate buffer saline (PBS, Sigma Aldrich) to remove medium constituents. Finally, cell pellets were redispersed in autoclaved water and diluted to a cell concentration of 10⁷ CFU/ml. Bacteria at concentrations of 10⁷ CFU/ml were incubated with different concentrations of nanomaterials (40-150 μg/ml) for 4 hours. After incubation, bacteria were plated in agar plates using serial dilution method and allowed to grow overnight.

SEM and TEM Analysis of Bacteria

For TEM, samples were initially fixed with 2.5% glutaraldehyde overnight at 4° C. and pelleted into 0.8% aggregate to form dense cell aggregates. The cell pellet was treated for 2 hours with 1% osmium tetraoxide in DPBS, followed by washing with deionized water and en-block stained overnight at 4° C. with aqueous 0.5% uranyl acetate. The cell pellet was dehydrated in series in a graded acetone series. The sample was sectioned and post stained using 2% uranyl acetate in 50% ethanol solution and Sato's lead citrate for 3-4 minutes. Images were acquired using Phillips CM-12 TEM operated at 80 kV using a Gatan model 791 side-mount CCD camera.

For SEM, samples were initially fixed using 2.5% glutaraldehyde at 4° C., followed by washing with DPBS. The samples were postfixed with 1% osmium tetraoxide in DPBS, followed by washing with deionized water and dehydration in graded ethanol series. The samples were critically dried, sputtered coated with gold-palladium and images was captured using JEOL JSM6300 SEM operated at 15 kV.

Membrane Potential Assay

The membrane potential of bacteria was determined following the manufacturer's protocol using a Baclight membrane potential kit (Invitrogen). Briefly, bacteria were harvested at mid-log phase and diluted to ˜10⁶ CFU/ml in autoclaved water. The bacteria were treated with different concentrations of MoSe₂-ssDNA (1×MBC, 0.5×MBC, 0.25×MBC and 0.125×MBC) for 4 hours. A fully depolarized sample was prepared on addition of 5 mM proton ionophore, carbonyl cyanide 3-chlorophenylhydrazone (CCCP). After treatment, samples were incubated with 30 mM DiOC₂ for 1 hour. Membrane potential was determined using Stratedigm S1300EXi cell analyzer equipped with A600 high-throughput autosampler, as ratio of cell exhibiting red fluorescence to cell exhibiting green fluorescence. Cell populations were gated based on measurements from untreated (polarized) and CCCP treated (depolarized) samples.

ROS Production Assay

E. coli and S. aureus were inoculated in LB and TSB medium, respectively, harvested at mid-log phase, and diluted to ˜10⁶ cells in autoclaved water. The samples were treated with different concentrations of MoSe₂/T₂₀ (SEQ ID NO: 2) (1×MBC, 0.5×MBC, 0.25×MBC) for 4 hours. After incubation, cells were stained using CellROX orange reagent (Invitrogen) following manufacturer's protocol. Briefly, samples were stained with 750 mM of CellROX orange reagent, and samples was analyzed using Stratedigm S1300EXi cell analyzer equipped with an A600 high-throughput autosampler and mcherry fluorescence output was used to determine the oxidative stress of the cells.

### Example 2—Eradication of Fungi Using MoSe₂/Chitosan Preparations

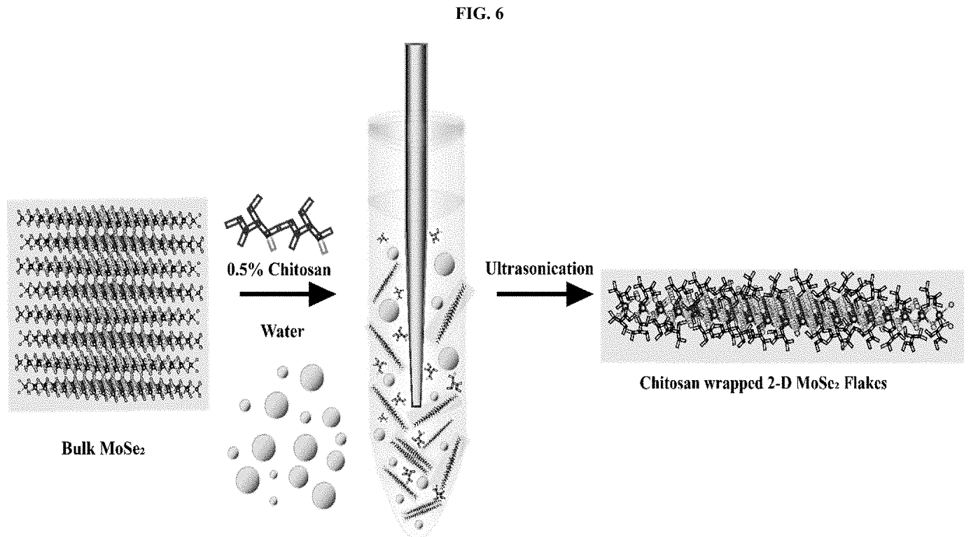

Chitosan (CS) is a linear polysaccharide is composed of distributed β-(1-4)-linked D-glucosamine and N-acetyl-D-glucosamine, which acts as a bulky group keeping the nanosheets from reaggregating due to steric repulsion, whereas the amine group (—NH₂) groups acts as the hydrophilic outer layer on the external side in the aqueous solution. Bulk MoSe₂ was dispersed in 0.5% low molecular weight (CS) in 1% acetic acid with the help of ultrasonication (FIG. 6).

Characterization:

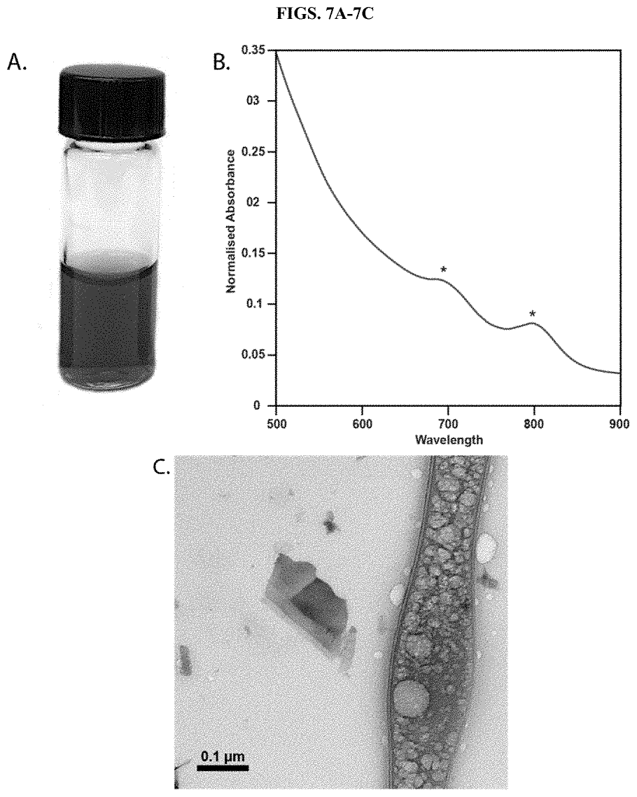

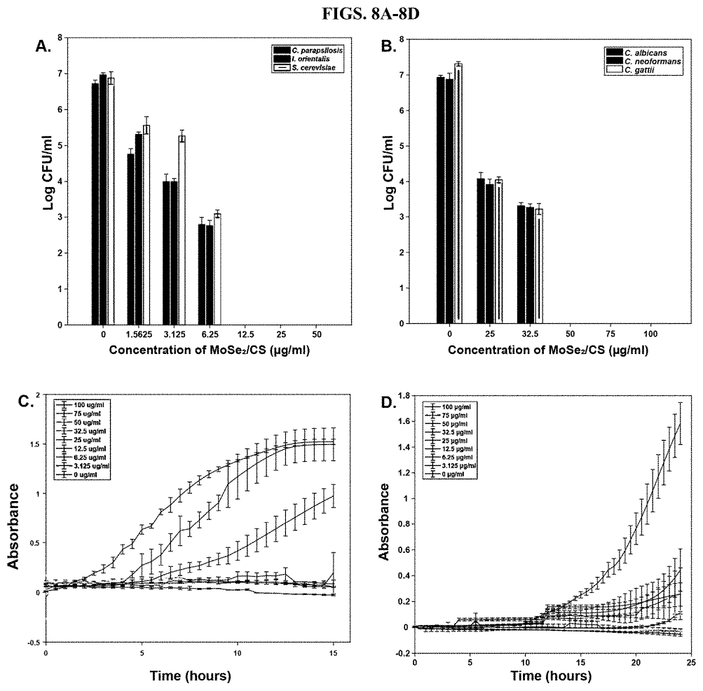

Visible to near-infrared spectroscopy was performed to identify the characteristic peaks of MoSe₂ at 700 nm and 800 nm (FIG. 7B). The dispersion has a similar appearance as MoSe₂/PLL/F77, with a dark brown color, and having a concentration of ˜0.32 mg ml⁻¹, as determined by ICP-MS (FIG. 7A). To ensure the cationic charge, zeta potential was measured for the samples with value ranging from +32 mV to +52 mV. TEM images displayed the 2D nature of the sheets with few- to mono-layer thickness flakes having lateral dimension ˜ 70 nm by −200 nm (FIG. 8C).

Antifungal Activity of MoSe₂/CS:

Fungi can be classified into two categories: unicellular fungi include I. orientalis, S. cerevisiae, C. parapsilosis, C. albicans, C. neoformans, and C. gattii; and filamentous fungi include A. fumigatus. CS is a known antifungal cationic biopolymer and successfully exfoliated MoSe₂ in aqueous medium with concentrations up to ˜0.32 mg ml⁻¹. MFC and MIC were calculated to determine the minimum fungicidal and inhibition concentrations of MoSe₂/CS, respectively. MFC of biosafety level 1 (BSL-1)I. orientalis, S. cerevisiae and C. parapsilosis was 12.5 μg ml⁻¹ (FIG. 8A). More resistant pathogenic BSL-2 C. albicans, C. neoformans, and C. gattii were less susceptible with MFC at 50 μg ml⁻¹ (FIG. 8B). MIC was performed on unicellular fungi C. albicans and filamentous fungi A. fumigatus. Results show inhibition of C. albicans at 12.5 μg ml⁻¹ and A. fumigatus at 32.5 μg ml⁻¹ (FIGS. 8C, 8D). Due to the filamentous nature and lack of individual colonies, microdilution test was not performed on A. fumigatus. The killing efficiency of MoSe₂/CS was compared against only 0.5% CS as control. Even though only 0.5% CS showed some killing, MoSe₂/CS was remarkably potent and more effective against all the above strains. This supports the synergistic killing mechanism where electrostatic interactions between the positively charged CS and negatively charged fungal cell wall bring the MoSe₂ nanosheets into close contact. The sharp edges of 2D MoSe₂ nanosheets perforate the cell wall to cause membrane damage. Then the CS can pass through the damaged cell membrane causing metabolic disruption and hindering oxidation processes inside the cytoplasm leading to cell death. The thin sharp edges of 2D MoSe₂ nanosheets and the positive charge of cationic CS thereby work synergistically to help damage fungal cell walls, leading to apoptosis.

Change in Cell Morphology:

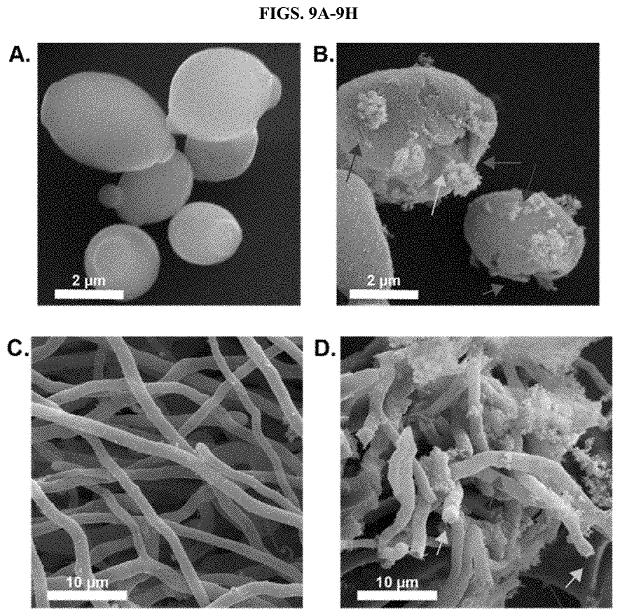

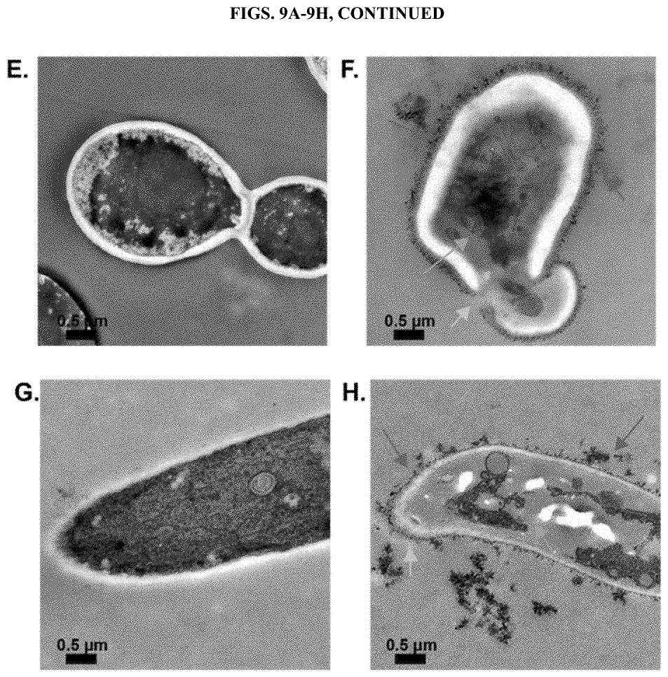

To observe the change in cell morphology after exposing the fungal cells to MoSe₂/CS, TEM and SEM were performed on unicellular fungi C. albicans and filamentous fungi A. fumigatus. It was compared with control samples that were subjected to the same treatment conditions in absence of MoSe₂/CS. A stark difference was observed between the two. SEM showed intact unicellular cells of C. albicans and healthy filaments of A. fumigatus (FIGS. 9A, 9C). Whereas, treated samples showed distinct membrane damage, breaking of filaments and deformed cells (FIGS. 9B and 9D). The cross-sectional view in TEM images of control samples showed intact cytoplasm with unbroken membrane and healthy cells (FIGS. 9E, 9G). The treated samples showed sharp-edged MoSe₂/CS nanosheets assembling around the fungal cell and filaments, broken outer cell wall and leaking of cytoplasm leading to apoptosis (FIGS. 9F, 9H). Thus, it can be concluded that the positively charged nanosheets/CS complexes localize around the negative outer membrane due to electrostatic interaction. This weakens the cell wall, destabilizing and reducing its rigidity, leading to disruption and membrane damage. The high turgor pressure inside the cell at the slightest disturbance enables the breaking of cell wall and cytoplasmic leakage. Hence, MoSe₂/CS synergistically weakens, damages, inhibits and kills unicellular as well as stronger filamentous fungi.

Biocompatability Tests:

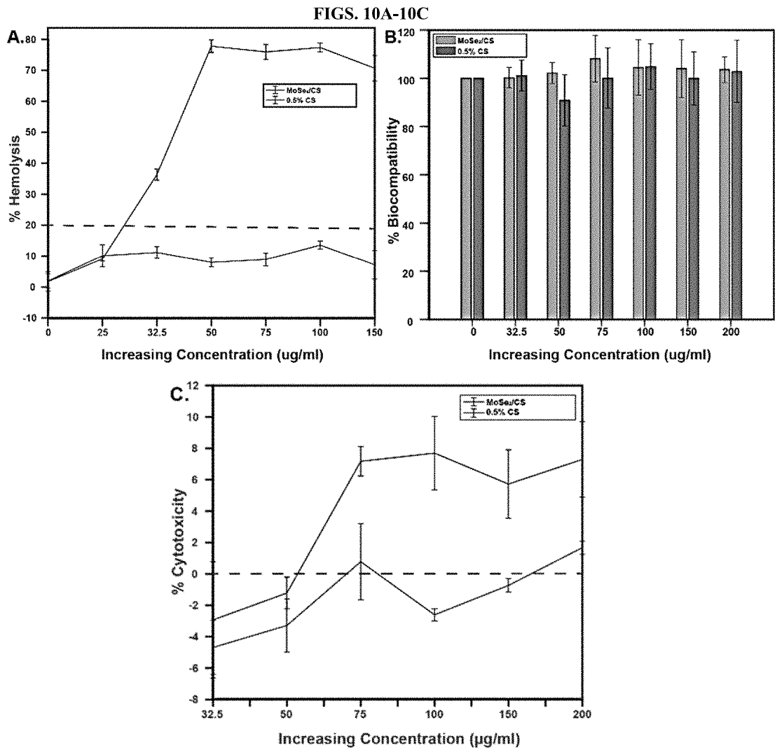

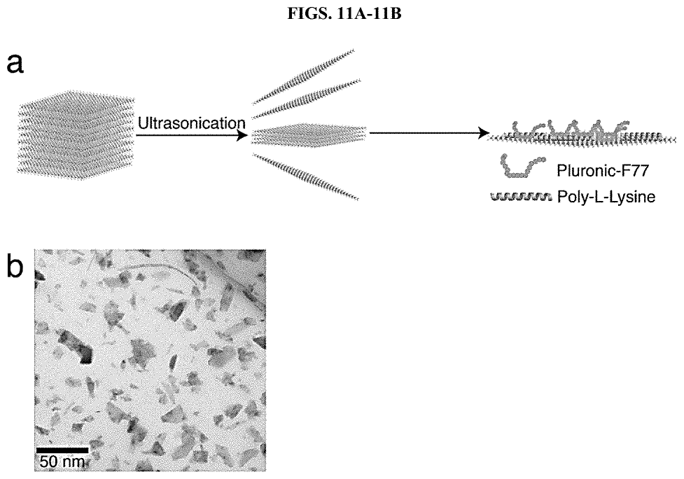

To test the biocompatibility, the hemolysis assay was performed by incubating different concentrations of MoSe₂/CS and only 0.5% CS with red blood cells (RBC) for comparison. After incubation for 3 h, ˜10%-12% lysing of RBCs was observed for MoSe₂/CS while far greater lysis of ˜78%-80% was observed for the same concentrations of 0.5% CS (FIG. 10A). In literature, 5% to 25% lysis is viewed to be biocompatible in in vitro assays. Subsequently, viability of mammalian cell line HEK 293 was tested using the alamarBlue assay in response to MoSe₂ dispersions. These results indicate that even after incubation for 4 hours with MoSe₂/CS at concentrations ranging from 0 μg ml⁻¹ to 250 μg ml⁻¹, more than 90%-100% of cells were viable, thus demonstrating MoSe₂/CS to be biocompatible (FIG. 10B). LDH assays were performed to estimate the cytotoxicity of the dispersions. MoSe₂/CS shows cytotoxicity to be below 1% compared to ˜8% cytotoxicity of 0.5% CS (FIG. 10C). Hence, the above results not only concur with each other in proving MoSe₂/CS to be a biocompatible nanomaterial but also shows it as less toxic than CS on its own, albeit at higher concentrations.

Materials and Methods:

Preparation of MoSe₂ Chitosan (CS) dispersions: A 0.5% solution of chitosan (CS) (Sigma-Aldrich) was made by dissolving the biopolymer in 1% acetic acid. 500 mg of bulk MoSe₂ and MoS₂ powders were taken and ultrasonicated in 20 mL of 0.5% chitosan for 2 hours at 25 W power using a Branson Digital Sonifier SFX 550. The sonicated sample was centrifuged at 5,000 rcf for 10-15 minutes followed by centrifuging at 21,000 rcf for 5 minutes using an Eppendorf 5424 Microcentrifuge. The supernatant was collected leaving the pellet containing excess nanomaterial and chitosan behind. For control experiments, 0.5% chitosan solution was sonicated and centrifuged following the same protocol.

Optical characterization and electron microscopy: UV-Vis spectra for the dispersed 2D MoSe₂/CS was acquired using a Jasco V-670 Spectrophotometer. A quartz cell with a path length of 1.0 cm was used for the measurements. TEM samples were prepared by drop-casting 10 μl of dilute dispersion on a holey carbon grid, which was dried under ambient conditions. Thereafter, the images were acquired on a Philips CM-12 TEM operated at 80 kV, with the help of a Gatan model 791 CCD camera.

Fungal cell preparation: Overnight cultures of Issatchenkia orientalis (I. orientalis), Saccharomyces cerevisiae (S. cerevisiae), and Candida parapsilosis (C. parapsilosis) were grown in yeast medium (YM). Candida albicans (C. albicans), Cryptococcus neoformans (C. neoformans), Cryptococcus gattii (C. gattii), and Aspergillus fumigatus (A. fumigatus) were grown in Sabouraud dextrose broth (SDB). Cells were harvested at 30° C. and 37° C. respectively. They were grown to mid-exponential growth phase (OD600=0.4). Cells were centrifuged at 2500 rpm for 5 mins and the pellets were washed with 1×PBS. The final pellet was resuspended in their respective growth medium and OD was measured. Cells were then diluted to 10⁶ CFU ml⁻¹. All experiments were performed in triplicate.

Measurement of minimum inhibitory concentration (MIC): The MIC values of MoSe₂/CS were determined using the same concentrations of nanomaterials used for MFC measurements. Equal volumes of fungal cell cultures at a concentration of 10⁵ CFU ml⁻¹ and MoSe₂/PLL/F77 were incubated at 37° C. in a 96-well plate in their respective growth medium. The optical density readings of each well at 600 nm were measured as a function of time using a microplate reader for over 12 hours in 30-minute intervals. Negative controls containing cells without nanomaterials were measured in parallel. Optical density was plotted against nanomaterial concentration, to determine the lowest concentration at which the optical density reading remains constant, i.e. no further growth was observed overtime. This concentration is defined as the MIC. All experiments were performed in triplicate.

Biocompatibility of MoSe₂/CS: The cytotoxicity of MoSe₂/CS toward HEK 293 cells was evaluated by Alamar blue assay and LDH assay. Cells were seeded in 96-well microplates at a density of 1×10⁵ cells ml⁻¹ in a 200-μl volume with DMEM medium. After 24 hours of cell attachment, the plates were washed with DPBS and the MoSe₂/CS at different concentration were incubated with the mammalian cells for 4 h and 24 h. Thereafter, wells were washed three times with 1×DPBS to remove any unattached cells. To check the viability of the attached cells, they were incubated with 200 μl of 10% alamarBlue solution in DMEM at 37° C. for 5 h. The fluorescence intensity was measured at 530 nm (excitation) and 590 nm (emission) using a microplate reader. To determine the cytotoxicity of MoSe₂/CS through damaged cells, supernatant from each well were pipetted out into a 96-well plate for LDH assay. 50 μl of supernatant and 50 μl of reaction mixture were incubated at room temperature for 4 h. The absorbance was measured at 490 nm and 680 nm using a microplate reader. Cell damage was expressed as a relative absorbance relative to that of Triton X 100 as a negative control and DMEM medium as a positive control.

Hemolysis assay: Fresh sheep red blood cells (RBCs) were diluted 1:20 in PBS (pH 7.4), pelleted by centrifugation, and washed three times in PBS (1,000 rcf, 10 min). The RBCs were counted using a cell counter and diluted to a final concentration of 2×10⁷ cells ml⁻¹. Equal volumes of RBC solution were incubated with varying concentrations of MoSe₂/CS sample solutions were incubated into a flat-bottomed 96 well plate in a humidified atmosphere containing 5% CO₂ at 37° C. for 3 hours. Following incubation, the 96 well plate was centrifuged (1,000 rcf, 10 min) and 100 μl of supernatant were transferred to a black 96-well plate. Hemoglobin release upon lysis of the RBCs were monitored through the optical absorbance at 405 nm (A405) using a microplate reader. Positive and negative controls for hemolysis were taken as RBCs lysed with 1% Triton X 100 (1:1 vol/vol) and RBC suspension in PBS, respectively. The percentage of hemolysis was calculated using the formula: % Hemolysis=[(A405 test sample −A405 negative control)/(A405 positive control −A405 negative control)]×100. The percent hemolysis was plotted against nanomaterial concentration, and the experiment was performed in triplicate.

Fungal cell morphology observation: For TEM, samples were initially fixed in suspension with 2.5% glutaraldehyde in DPBS overnight at 4° C., followed by washing in DPBS. Cells were then pelleted into 1% agarose and treated with 1% osmium tetroxide in DPBS for 1 hour, washed thoroughly with deionized water, and dehydrated in a graded acetone series. Spurr's epoxy resin was used to infiltrate and embed the samples. 70-nm sections were cut on a Leica Ultra cut-R microtome followed by post-staining with uranyl acetate and lead citrate. Images were generated on a Philips CM-12 TEM operated at 80 kV and acquired with a Gatan model 791 CCD camera. For SEM, cells were initially fixed and washed following the same method used for TEM samples. Washed cells were then concentrated into a small volume of DPBS and applied to PLL-coated coverslips for 10 minutes. Excess cells were removed by briefly rinsing in DPBS and the coverslips were transferred to a solution of 1% osmium tetroxide in DPBS for 1 hour, followed by thorough washing with deionized water. Samples were dehydrated in a graded ethanol series and critical-point dried in a Balzers-Union CPD-020 unit using carbon dioxide as the transition fluid. After routine mounting on aluminum stubs, samples were sputtered-coated with 10-12 nm of gold-palladium in a Technics Hummer-II unit. Images were generated on a JEOL JSM6300 SEM operated at 15 kV and acquired with an IXRF model 500 digital processor.

### Example 3—Eradication of Bacterial Biofilms Using MoSe₂/PLL/F77

The increasing pervasiveness of infections caused by multidrug resistant (MDR) bacteria is a major global health issue, which has been further exacerbated by the limited number of effective antibiotics introduced in last 40 years. Drug-resistant bacteria lead to significant morbidity and mortality, with 1.7 million MDR infections and nearly 100,000 deaths from these infections each year in the United States and have wide ranging clinical effects in surgery, premature infant care, cancer chemotherapy, and transplantation medicine. The traditional strategy for combating these drug-resistant superbugs involves the development of new antibiotics, but the two classes of antibiotics approved for clinical use over the past two decades have not exhibited activity against a broad spectrum of bacteria. Furthermore, the mutation-prone replication machinery of bacteria enables resistance to develop rapidly against therapies that rely on a single mechanism of action, allowing clinically significant resistance to appear within a few months of their introduction. For examples, Ceftaroline clinical use began in 2010 and, less than a year later, resistance was observed in patients with Neisseria gonorrhoeae, Enterobacter, and Staphylococcus aureus infections. Thus alternative strategies are needed to combat the rise of MDR bacteria.

Unlike traditional antibiotics that target intracellular targets in bacteria, next-generation strategies for drug-resistant bacteria have involved the development of antibiotics with the ability to circumvent the resistance-developing machinery of bacteria, alternative antimicrobial actions, and ability to eliminate multiple strains of bacteria. Promising alternative strategies in these regards include antimicrobial peptides (AMPs) and their related analogues (Gupta et al., Journal of the American Chemical Society 140:12137-12143, 2018) and nanoantibiotics (Gupta et al., Chemical Society Reviews 48:415-427, 2019). AMPs are short cationic peptides of that provide an innate defense mechanism in all living organisms. They hold the ability to specifically target bacterial cell membranes using electrostatic interactions, leading to disruptions of cell membranes and finally cell death. Despite their success in eliminating a broad spectrum of drug-resistant bacteria, the implementations of AMPs and related analogues in healthcare settings is significantly hindered by their tendency to interact with mammalian cells, which leads to significant toxicity. Furthermore, the high production costs and tendency to undergo degradation in the presence of proteases, further hinder their use as antibiotics. Nanoantibiotics rely on nanotechnology for the development of antibacterial materials, including nanoparticles, metal and metal oxides, carbon-based materials, and surfactant-based emulsions for eradication of superbugs. The high surface-to-volume ratio and unique physico-chemical properties of nanomaterials are the two key factors that contribute to their ability to thwart multidrug-resistant bacteria. Furthermore, multiple antibacterial mechanisms of nanoantibiotics such as the disruption of bacterial cell membranes and generation of oxidative stress, make them effective against multiple strains of bacteria and inhibits the resistance-development machinery. Despite their promise as antibacterial candidates, the high toxicity and challenges of formulating them into effective drugs hinder their further use for biomedical applications. Also, most of the reported antimicrobial systems are highly efficient against planktonic bacteria, while they fail to function against biofilms. Nanotechnology-enabled antibacterial systems that are highly effective against both planktonic bacteria and biofilms and remain easy to construct and biocompatible towards mammalian and red blood cells cell remain much needed tools for treating MDR infections.

To overcome these problems, two antimicrobial components, AMPs and nanoantibiotics, were integrated into a single system to synergistically to combat MDR bacteria. In this section, the engineering of a new class of antimicrobial agents consisting of cationic polypeptides, poly-L-lysine (PLL) encapsulating two-dimensional transition metal dichalcogenides, MoSe₂ is described. The incorporation of polypeptide, PLL on nanomaterial surfaces reduces non-specific peptide interactions with mammalian cells while facilitating specific interactions with negatively charged, bacterial cell membrane. It was hypothesized that the presence of cationic peptides on the surface of MoSe₂, a two-dimensional material with intrinsic antibacterial activity, would increase the local concentrations of the cationic peptides and enable them to impart stronger disruptive effects on the bacterial cell membrane. For further stabilization in high salt concentrations of biological systems, we further incorporated the non-ionic block copolymer Pluronic F77 to provide steric stabilization of the MoSe₂/PLL complex. The designed MoSe₂/PLL/F77 systems proved highly efficient in eradicating both gram-positive and gram-negative ‘ESKAPE’ strains, with minimum bactericidal concentrations (MBCs) lower than 50 μg/ml. Furthermore, MoSe₂/PLL/F77 systems showed minimal to no toxicity towards both red blood cells and mammalian cells. Our studies also revealed that MoSe₂/PLL/F77 can successfully penetrate and eradicate biofilms, while maintaining biocompatibility towards mammalian cells. Also, when tested in co-culture systems, MoSe₂/PLL/F77 showed 100% eradication of planktonic bacteria and 3-log reductions of biofilms while posing no toxicity towards mammalian cells. Unlike conventional antibiotics, MoSe₂/PLL/F77 did not show any significant generation of resistance towards gram-positive S. aureus and gram-negative P. aeruginosa after 20 serial passages. Furthermore, mechanistic evaluation of the antibacterial actions of MoSe₂/PLL/F77 demonstrated a multimodal antibacterial mechanism that includes electrostatic interactions with bacterial cell membrane, followed by disturbance to membrane potential, oxidative stress, and finally cell death.

Inspired by previous reports of the antibacterial activity of naturally occurring cationic peptide, commercially available cationic polypeptide, poly-L-lysine (PLL), was selected. The amphipathic cationic nature of PLL performed a dual function: first, its positive charge promoted interactions with negatively charged bacterial cell membrane, and second, its hydrophobic butyl chains enabled it interact with the surface of the two-dimensional transition metal dichalcogenide MoSe₂. MoSe₂ was ultrasonicated in presence of PLL in a weight ratio of 20:1 for 2 hours (FIG. 12A). During this process, the cationic polypeptides undergo intercalation between layers of MoSe₂, weakening the van der Waals interactions, and assisting the exfoliation of MoSe₂; whereas the presence of cationic polypeptides on the surface of MoSe₂ further stabilizes the nanosheets in solution via electrostatic repulsion. The zeta potential of PLL-dispersed MoSe₂ was +41 mV, which indicated presence of electrostatic repulsion as a key factor towards stabilization of flakes in colloidal dispersion.

Despite the high stability of the PLL-dispersed MoSe₂ in colloidal suspensions, PLL dispersed MoSe₂ aggregated in buffer media containing high salt concentrations, since these conditions led to shielding of the electrostatic repulsion between nanosheets. To further stabilize the system in buffer media, we complemented PLL on the surface of the nanosheets with the block copolymer Pluronic F77, which provided additional stabilization to the nanosheets using steric repulsion. Zeta potential measurements of the colloidal dispersion showed a reduction of zeta potential to +21 mV, indicating replacement of some of the adsorbed PLL by Pluronic F77 on the MoSe₂ surface. The resulting MoSe₂/PLL/F77 hybrid structure was found to be stable in a wide range of buffers, including M9, MEM and 1×PBS. After successful stabilization of MoSe₂ nanosheets using PLL and Pluronic F77, excess polymer was removed from the solutions using dialysis. After purification, the MoSe₂/PLL/F77 dispersion was characterized using UV-Vis spectroscopy, which showed the presence of two excitonic peaks. The presence of the excitonic peaks for dispersed solutions, demonstrated the pristine nature of MoSe₂ after exfoliation. We further evaluated the morphology of nanomaterial using transmission electron microscopy (TEM) and atomic force microscopy (AFM). The morphological analysis of MoSe₂/PLL/F77 using TEM, demonstrated successful exfoliation and thin-flake like structure of dispersed nanomaterial with lateral dimensions in the range of 50-100 nm (FIG. 12B). The polymer content on the surface of MoSe₂ was further determined using thermogravimetric analysis (TGA). The TGA analysis of MoSe₂/PLL/F77 demonstrated the presence of 22 wt % polymer on the surface of MoSe₂.

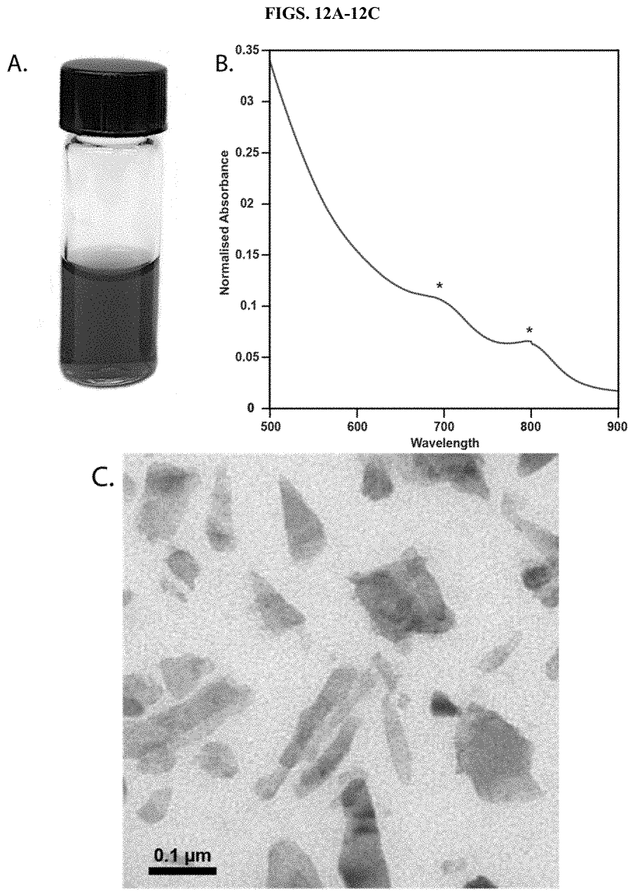

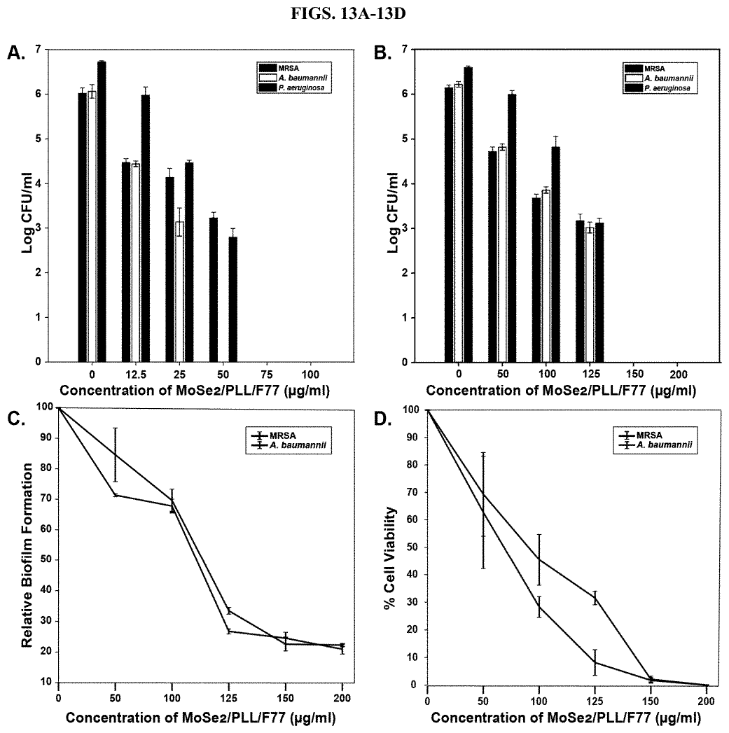

Further 2D MoSe₂/PLL/F77 preparation and characterization: MoSe₂/PLL/F77 was prepared via ultrasonication of bulk molybdenum diselenide (MoSe₂) powder (Sigma-Aldrich) in solution phase in a two-step process using 2 mg ml⁻¹ PLL followed by 0.5% Pluronic F77. The resulting dispersion has a dark brown color (FIG. 12A). Visible-near-infrared spectroscopy (UV-vis-NIR) and transition electron microscopy (TEM) were used to characterize the structure and composition of MoSe₂/PLL/F77. The concentration MoSe₂/PLL/F77 was ˜0.3 mg ml⁻¹ determined using ICP-MS. Vis-NIR spectra of the MoSe₂ dispersions were acquired in the range of 500-900 nm at room temperature. Characteristic adsorption peaks for excitonic transitions were observed at 700 nm and 800 nm marked by asterisks “*” in FIG. 12B. The zeta potential was +38 mV, where the positive charge of PLL stabilizes the sheets through electrostatic repulsion. TEM measurements indicate the biopolymer dispersions contained thin nanosheet structures (FIG. 13C). Typical lateral sizes of the flakes were ˜50 nm by ˜150 nm for MoSe₂.

Antibacterial Activity:

Effect of MoSe₂/PLL/F77 on bacterial biofilms: The minimum bactericidal concentration (MBC) was determined against MRSA, A. baumannii and P. aeruginosa. MBC is the minimum concentration of material at which complete death of bacterial cells is observed in solution medium. Minimum biofilm eradication concentration (MBEC) on the other hand determines the minimum concentration of material required to eradicate an existing biofilm. MBC and MBEC were determined using microdilution tests in TSB medium. MRSA and P. aeruginosa showed MBC at 75 μg ml⁻¹, whereas A. baumannii was slightly more susceptible towards MoSe₂/PLL/F77 with MBC at 50 μg ml⁻¹ (FIG. 13A). Additional studies also showed that the MoSe₂/PLL/F77 provide an MBC of 50 μg ml⁻¹ against MDR isolates of all of the ‘ESKAPE’ strains (E. faecalis, S. aureus, K. pneumoniae, A. baumannii, P. aeruginosa, Enterobacter). We also performed a multi-day study to determine if MDR P. aeruginosa and S. aureus could evolve resistance to the MoSe₂/PLL/F77 at 0.5×MBC concentrations administered over a set of 20-consecutive 16-hour passages. In the two-week period, no increase in MBC was observed for either strain, while resistance quickly developed over only a few days when the cells were exposed to antibiotics and PLL using analogous procedures.

To determine MBEC, biofilm of each strain was grown on a 96-well plate for 48 h and treated with different concentrations of MoSe₂/PLL/F77 ranging from 50 μg ml⁻¹ to 200 μg ml⁻¹. Results show that all three strains display MBEC at 150 μg ml (FIG. 13B). Formation of biofilms leads to a rigid hydrated extracellular polymeric substrate (EPS) secreted by bacteria, which is difficult for any particle or drug to penetrate. The EPS secreted varies in composition from strain to strain, but they are principally composed of DNA, lipids, and humic substances making them negatively charged. Owing to the negatively charged matrix covering the biofilm, the cationic MoSe₂/PLL/F77 is attracted to EPS, leading to interaction of negatively charged EPS and positively charged chitosan. Also, the 2D thin nature of MoSe₂ nanosheets can effectively perforate through the thick EPS layer to reach the cells underneath, causing membrane disruption and triggering cell death.

To quantify biofilm formation and viability, gram-positive MRSA and and gram-negative A. baumannii was used to perform crystal violet (CV) assay and XTT assay. CV is a basic dye consisting of hexamethyl pararosaniline chloride. It binds to the negatively charged molecules and stains the bacteria cell as well as the surrounding EPS. The results suggest that as the concentration of MoSe₂/PLL/F77 was increased the remaining biofilm mass decreases gradually. As the biofilm mass decreases to 25% of the mass of the untreated biofilm at 125 μg ml⁻¹ (FIG. 13C). It further decreases to 20% at 200 μg ml⁻¹. This supports the concept of electrostatic interaction between the positively charged MoSe₂/PLL/F77 and negatively charged EPS leading to detachment or uprooting of biofilm from the surface leading to mass loss.

The XTT assay is an effective way to determine the metabolic activity or the viability of bacterial cells. The colorless tetrazolium salt upon reduction, when in contact with undamaged cell surface, turns bright orange due to trans-membrane-plasma membrane electron transfer and indicates a metabolically active bacterial cell. The results agree with the CV assay showing drastic decrease in the metabolic activity of the bacteria with increases in MoSe₂/PLL/F77 concentration. The metabolic activity of the bacteria decreases to 7% at 125 μg ml⁻¹ and eventually to 0% at 200 μg ml⁻¹ (FIG. 13D). The results also indicate that, despite the residual biomass left as observed in the CV assay, there is little or no metabolic activity left in the biofilm. This points to the fact that even though biofilm is not completely removed due to electrostatic interaction, it is damaged and killed, reducing the metabolic activity to almost zero.

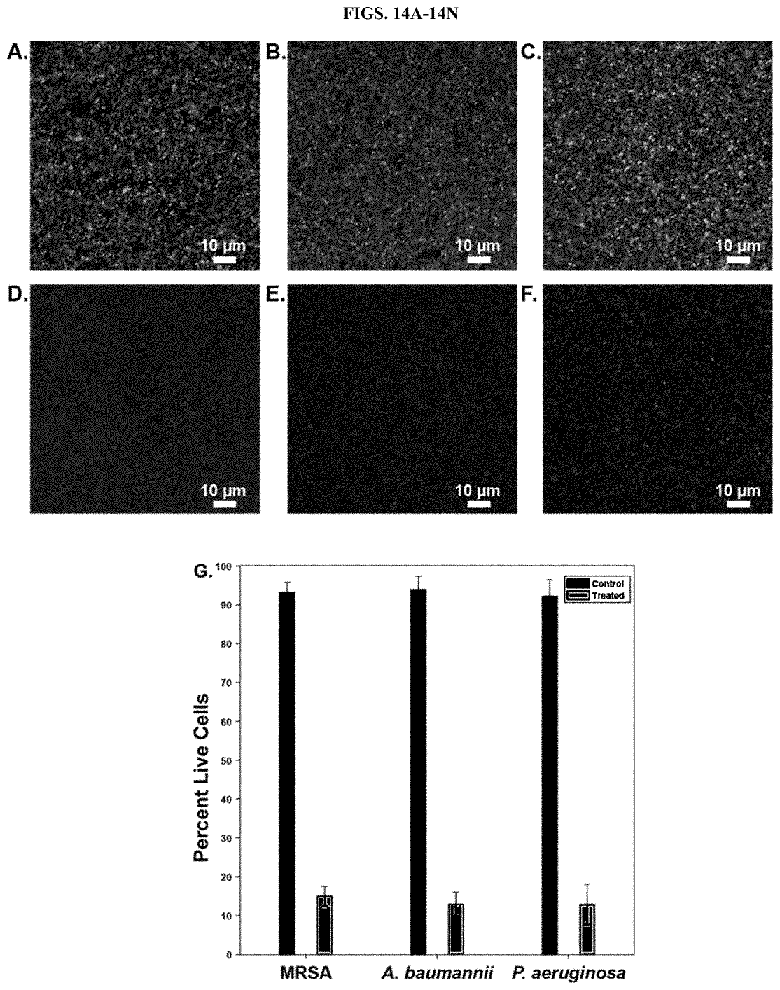

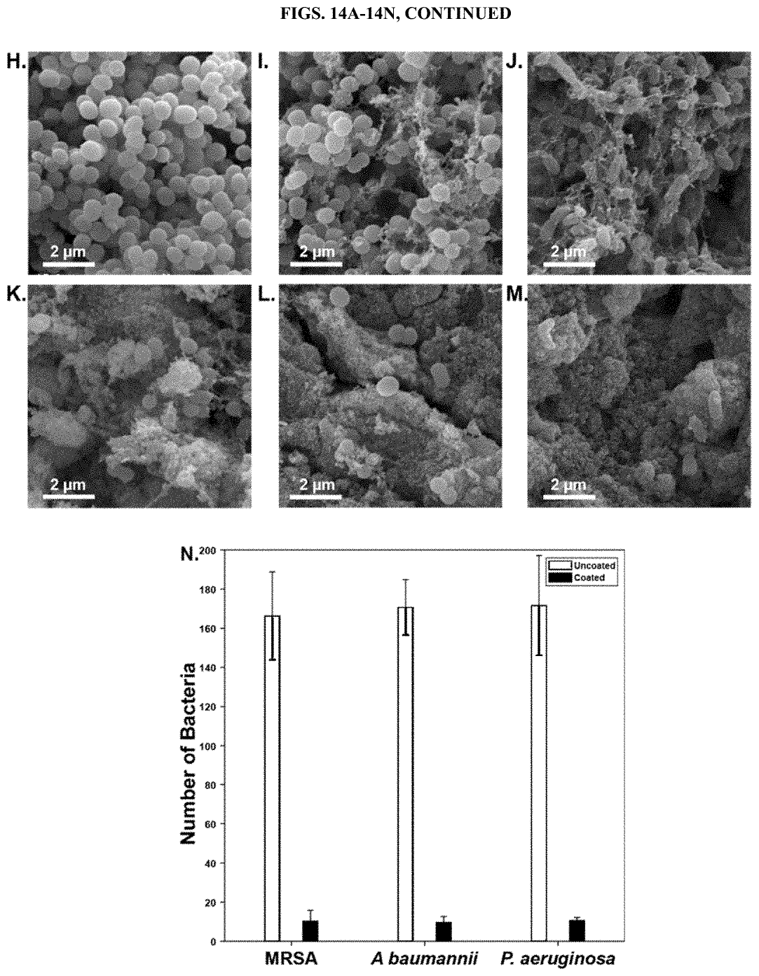

Killing and inhibition of biofilm growth: Confocal imaging was done to visualize the biofilm coverage and the live-to-dead cell ratio after treating with MoSe₂/PLL/F77. All three strains of bacteria were grown for 48 h to form a biofilm. Biofilm-containing control slides as well as slides treated with ˜150 μg ml⁻¹ of MoSe₂/PLL/F77 were treated with SYTO9 green-fluorescent nucleic acid stain and the red-fluorescent PI. SYTO9 can label the entire population of cells, both healthy and damaged. In contrast, PI can only penetrate bacteria with damaged membranes and also causes a reduction in SYTO9 stain fluorescence once it penetrates the cell. Thus, green fluorescence indicates live cells and red fluorescent indicates dead or damaged cells. From the confocal images (FIGS. 14A-14F), it is verified that control slide in absence of MoSe₂/PLL/F77 shows ˜95.49%, ˜94.63% and ˜89.2% percent of live cells for MRSA, A. baumannii and P. aeruginosa respectively. Whereas, the MoSe₂/PLL/F77 treated slides only show ˜14.55%, ˜12.15% and ˜14.17% viable cells (FIG. 14G). Thus, biofilms are damaged and killed by the presence of MoSe₂/PLL/F77.

Along with confocal imaging, SEM imaging was also done on control and treated samples to microscopically view the biofilm change. Biofilms were first grown on MBEC assay plates with stubs for 48 h followed by treatment with MoSe₂/PLL/F77 at a concentration 150 μg ml⁻¹. The samples were then processed to observe the change in biofilm morphology in the control compared to treated samples. Samples without MoSe₂/PLL/F77 showed thick layers of biofilm expanding over the surface (FIGS. 14H-14J) while the treated samples lacked healthy cells, biofilm, or EPS (FIGS. 14K-14M). After treating MRSA, A. baumannii, and P. aeruginosa biofilms with MoSe₂/PLL/F77, the SEM images showed only ˜6.89%, ˜6.58%, and ˜4.42% cells remaining compared to their control samples respectively (FIG. 14N). The cells had apparent deformities and images showed MoSe₂/PLL/F77 was wrapped around the cells, demonstrating damaging of cells and inhibition in proliferation of biofilm and EPS matrix, respectively.

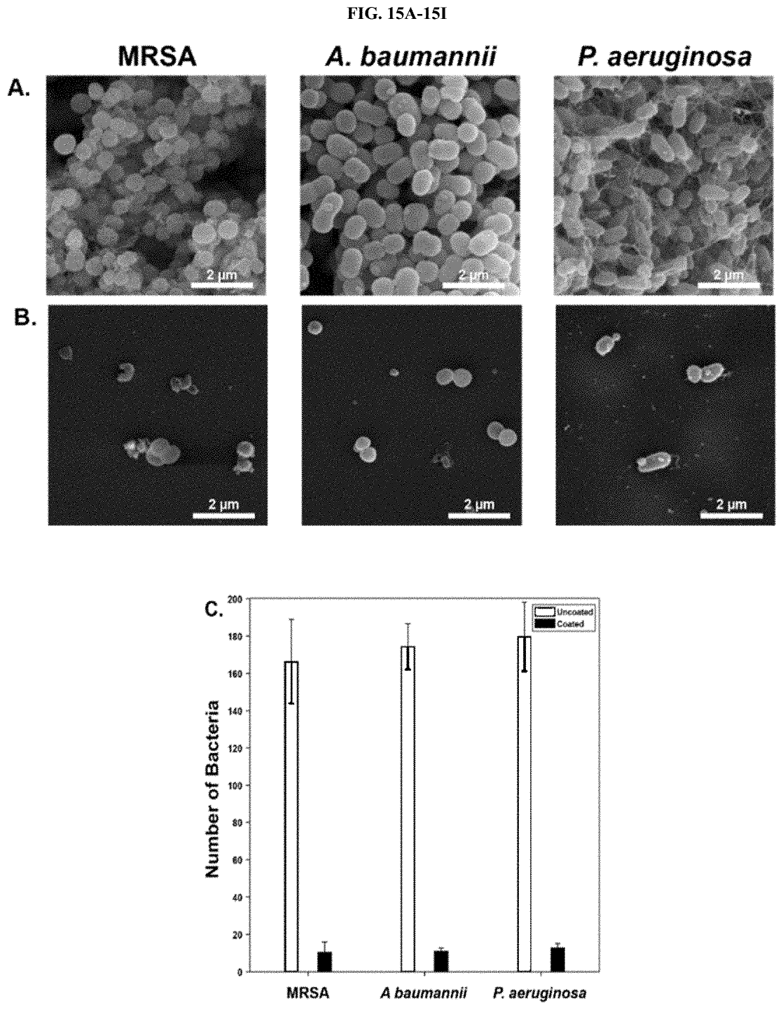

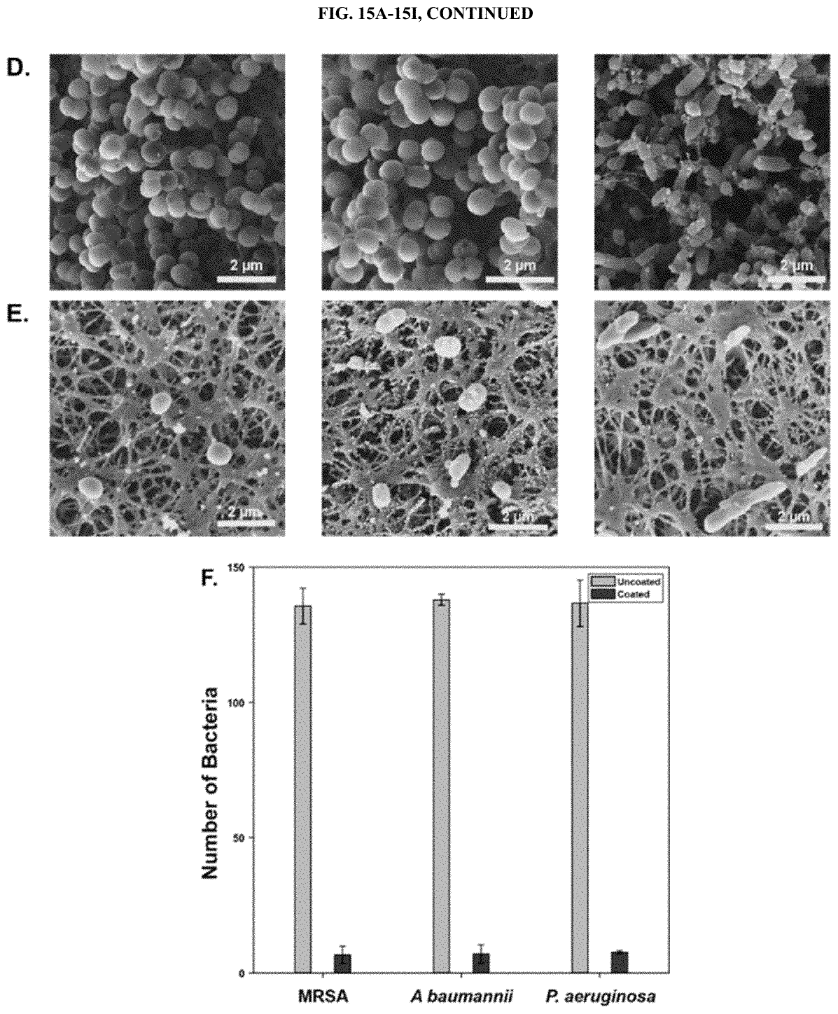

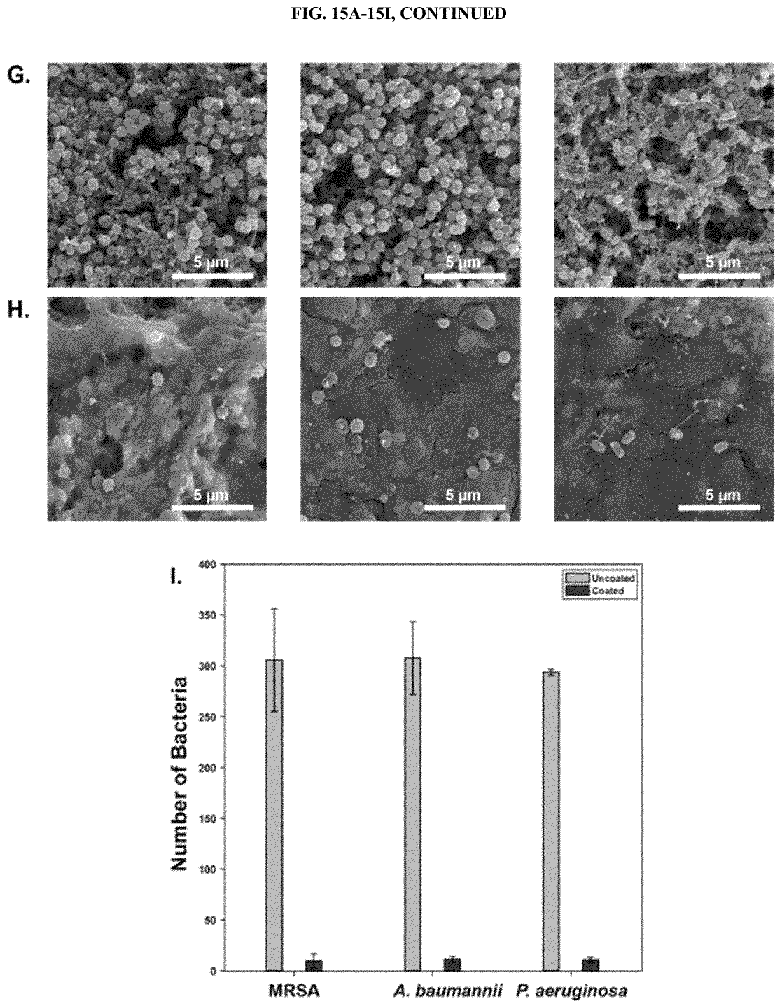

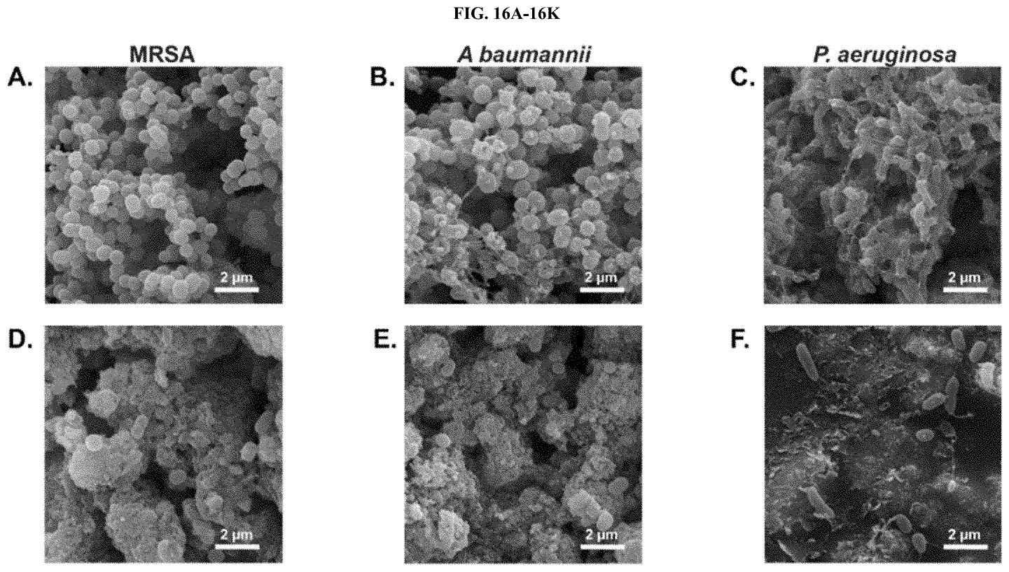

Coating substrate to inhibit biofilm growth: Biofilm growth and nosocomial infections can be caused by pathogenic bacteria harbored on medically instruments like implants, catheters, and pacemakers. Hence, we examined the capacity of surfaces coated with MoSe₂/PLL/F77 to deter bacterial growth. The medically relevant surfaces PMMA, which is used to coat denture strips; hydrophilic PTFE, which is used to coat catheters; and medical grade Ti alloy were coated with MoSe₂/PLL/F77 and exposed to bacteria. MRSA, A. baumannii, and P. aeruginosa biofilms were grown on uncoated (i.e., no MoSe₂/PLL/F77) and MoSe₂/PLL/F77-coated surfaces followed by SEM imaging to see the differences. All the coatings repressed cell growth with dramatic differences in the final biofilm formation between control surfaces and coated surfaces (FIGS. 15A, 15B, 15D, 15E, 15G, 15I). PMMA-coated glass slides showed only ˜6.53%, ˜5.39%, and ˜5.69% biofilm formation for MRSA, A. baumannii and P. aeruginosa, respectively, on coated samples compared to the uncoated samples (FIGS. 15A, 15B, 15C). Hydrophilic PTFE showed ˜3.93%, ˜5.02%, and ˜6.57% biofilm formation for MRSA, A. baumannii and P. aeruginosa, respectively, on coated samples compared to the uncoated samples respectively (FIGS. 15D, 15E, 15F). Medical grade Ti alloy had ˜3.28%, 4.30%, and ˜2.66% biofilm of MRSA, A. baumannii, and P. aeruginosa on coated samples compared to the uncoated samples respectively (FIGS. 15G, 15H, 15I).

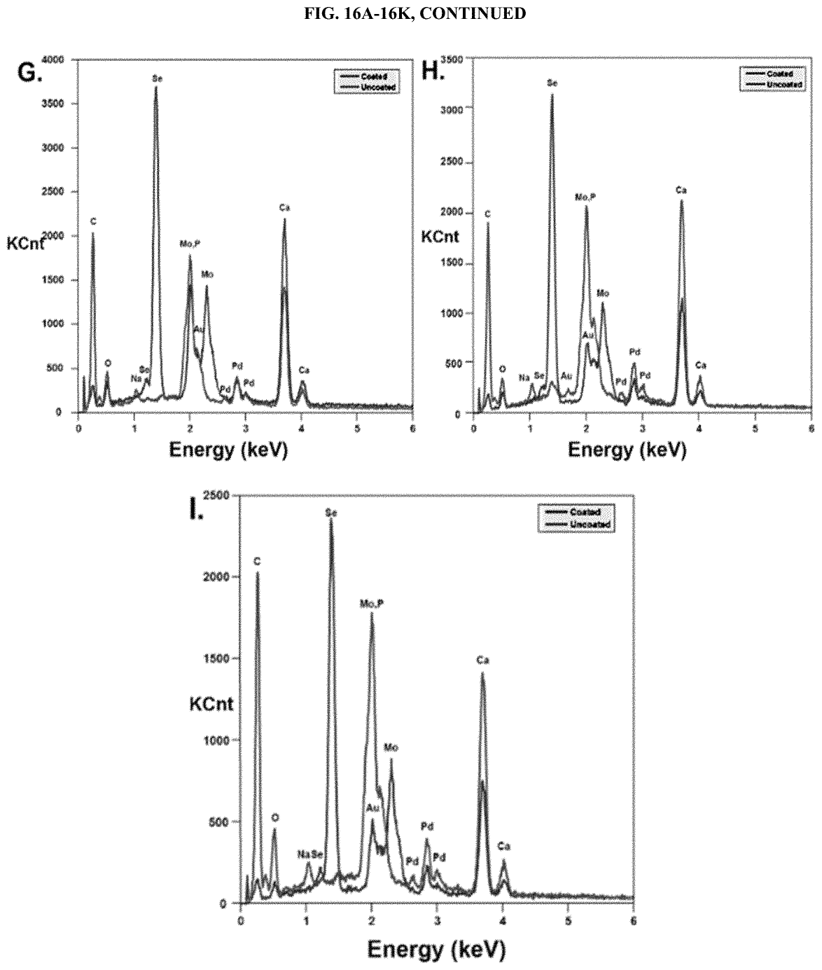

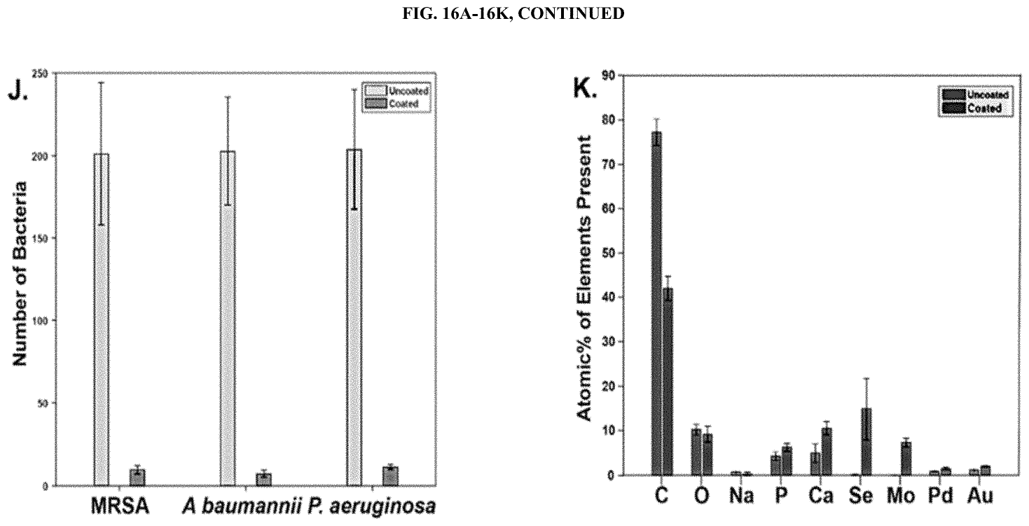

To demonstrate the efficiency of the coating, MoSe₂/PLL/F77 was coated on the lower half of the stub of an MBEC assay plate while the top half was kept uncoated. Subsequently, biofilm was growth was initiated along the entire stub for 48 h and processed for SEM imaging to observe the efficacy of the coating. Despite being on the same stub and treated under the same conditions, the uncoated part of the stub showed complete coverage by biofilm while the MoSe₂/PLL/F77-coated region had few individual cells to none present (FIGS. 16A-16F). MRSA, A. baumanni, and P. aeruginosa biofilms had ˜4.08%, 4.19%, and 5.84% biofilm formation on the bottom coated region compared to the top uncoated region (FIG. 16J). To ensure that the bottom region was coated with MoSe₂/PLL/F77, EDX was performed on the coated as well as the uncoated region and compared for all three strains MRSA, A. baumannii, and P. aeruginosa (FIGS. 16G, 16H, 16I). Both the uncoated (bottom) and coated (top) region showed presence of carbon (C), oxygen (O), sodium (Na), phosphorous (P) and calcium (Ca). EDX also had trace amounts of palladium (Pd) and gold (Au) from the sputter coating. Coated region showed strong presence of ˜8 and ˜17 atomic percent of molybdenum (Mo) and selenium (Se) respectively (FIG. 16K). While there was negligible Mo or Se present in the uncoated region. It was also observed that the percentage of C decreases significantly on the coated region to ˜40 atomic percent as opposed to ˜80 atomic percent in the uncoated region having extensive biofilm growth (FIG. 16K). This lack of biofilm in the coated regions of the same substrate reaffirms the fact that the coating of MoSe₂/PLL/F77 is inhibiting the growth of biofilm and killing bacteria coming into contact with its surface.

Thus, from the above results, it was concluded that MoSe₂/PLL/F77 coating was successful over a variety of medically relevant surfaces. Also, not only does it efficiently impede and hinder the growth of biofilms but in the process damages the EPS matrix resulting in biofilm eradication.

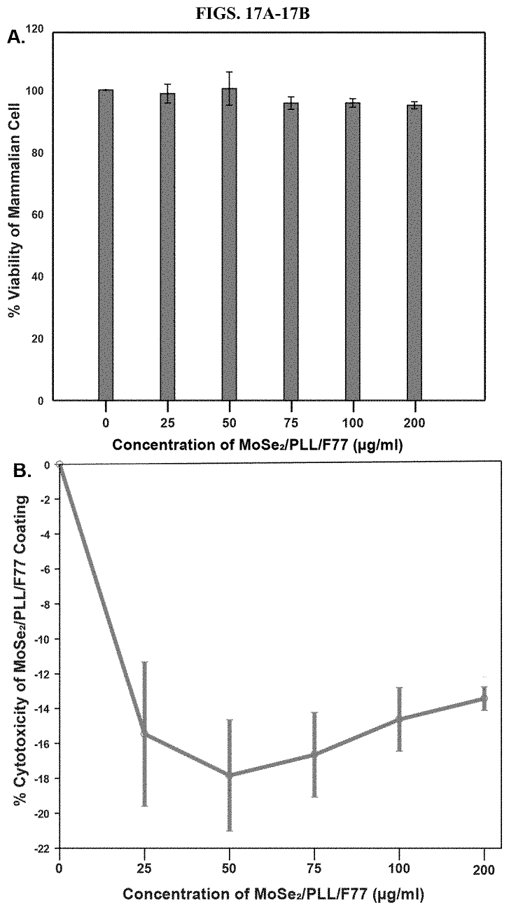

Biocompatibility test: In order to ensure the biocompatibility of these coated surfaces, the viability of mammalian HEK 293 cells were tested with alamarBlue assay and the cytotoxicity of the coating was observed with an LDH assay. Resazurin present in alamarBlue indicates oxidation-reduction that is demonstrated by a colorimetric change. The reduced resorufin gives a fluorescent pink color, the intensity is proportional to the percentage of viable cells respiring. Thus, the change in color indicates the oxidation due to respiration quantitatively measuring the viability of mammalian cells in presence of the coated substrate. The results show 98-100% viability of mammalian cells (FIG. 17A).

The supernatant was collected before treating the mammalian cells with alamarBlue followed by LDH assay. Lactate dehydrogenase is a cytosolic enzyme secreted by damaged mammalian cells. The secreted LDH can be quantified by catalyzing the enzymatic reaction where tetrazolium salt is converted to a red formazan product. The level of formazan is proportional to percent of damaged cells. The results show −18% cytotoxicity at 50 μg ml⁻¹ of MoSe₂/PLL/F77 and a −11% cytotoxicity at 200 μg ml⁻¹ (FIG. 17B). The lack of cytotoxic effect can be concluded from the negative cytotoxicity percentage, which indicates negligible LDH secretion and undamaged mammalian cells.

Methods and Materials

Preparation of MoSe₂/PLL/F77 dispersions:

The MoSe₂/PLL/F77 dispersions were prepared by probe sonicating 200 mg of MoSe₂ in the presence of 2 mg ml⁻¹ PLL in 8 ml of water. The sonication was done for 2 h at 25 W power using Branson Digital Sonifier SFX 550. The sonicated sample was centrifuged for 5 minutes at 5000 ref followed by 1 min at 21000 ref using an Eppendorf 5424 Microcentrifuge. The supernatant was collected leaving the pellet which contained excess MoSe₂ and poly-L-lysine (PLL). The MoSe₂/PLL solution was further sonicated in 0.5% Pluronic F77, an amphiphilic nonionic biocompatible surfactant for 30 mins at 11 W power. The resultant solution was then dialyzed for 36 h in water to remove excess the PLL and Pluronic F77. The concentration of nanosheets in the final dispersion was determined using ICP-MS. The MoSe₂/PLL/F77 dispersions were stable for several weeks.

Optical Characterization and Electron Microscopy:

UV-Vis spectra for the dispersed 2D MoSe₂/PLL/F77 was acquired using a Jasco V-670 Spectrophotometer. A quartz cell with a path length of 1.0 cm was used for the measurements. TEM samples were prepared by drop-casting 10 μl of dilute dispersion on a holey carbon grid, which was dried under ambient conditions. Thereafter, the images were acquired on a Philips CM-12 TEM operated at 80 kV, with the help of a Gatan model 791 CCD camera.

Biofilm Growth:

Overnight cultures of methicillin-resistant Staphylococcus aureus (MRSA), Acinetobacter baumannii (A. baumannii), and Pseudomonas aeruginosa (P. aeruginosa) were grown in tryptic soy broth (TSB) at 37° C. and harvested at mid-exponential growth phase (optical density (OD) at 600 nm wavelength, OD600=0.33). Bacteria samples were diluted in TSB medium to obtain an of OD600=0.01. 150 μl of this stock solution from each strain was incubated in 96-well plates or minimum biofilm eradication concentration (MBEC) assay plates coated with hydroxyapatite. Cells were were incubated at 37° C. for 48 h and the growth medium was changed after 24 h. All the experiments were done in triplicate.

Measurement of minimal bactericidal/fungicidal concentration (MBC): Different concentrations of MoSe₂/PLL/F77 were made by serial dilution. Bacterial cells were grown to OD600 of ˜0.4 and ˜0.33, respectively, and diluted to 10⁶ CFU (cell-forming units) ml⁴ in their respective growth medium. Equal volumes of cell culture and nanomaterial at different concentrations were then added to a 96 deep-well plate. The 96 deep-well plate was then incubated at 37° C. for 2 h. The final cell viability was determined using the microdilution method on agar plates. For MRSA, A. baumannii and P. aeruginosa agar plates were incubated overnight at room temperature and then at 37° C. with aeration for 4-6 h.

Minimum biofilm eradication concentration (MBEC): The 96-well plates were used to grow biofilms of MRSA, A. baumannii and P. aeruginosa as mentioned above. The biofilms were washed three times with 1×PBS, followed by incubation at different concentrations of MoSe₂/PLL/F77 solution. 1×PBS was used as positive control. It was treated for 6 h at 37° C. After treatment, the wells were pipetted gently to mix the biofilm adhered at the bottom of the 96 well plate. The MBEC was calculated using the microdilution method on TSB agar plates. The colonies were counted after 16 h to determine the MBEC.

Biofilm formation assay (Crystal violet): Biofilms of MRSA, A. baumannii, and P. aeruginosa were grown on 96-well plates as described above. Each film was washed three times in 1×PBS to remove unattached cells. The biofilm was incubated with different concentrations of MoSe₂/PLL/F77 solutions at 37° C. for 4 h. After incubation the MoSe₂/PLL/F77 solution was discarded and the 96-well plate was washed with 1×PBS. The plate was then incubated with 150 μl of a 0.1% crystal violet solution and incubated for 30 mins at room temperature. The plate was rinsed and washed with 1×PBS and kept upside down for it to dry out completely for 2-3 h. Each well was treated with 150 μl of 30% acetic acid for 30 mins at room temperature to dissolve the crystal violet attached to the biomass. 100 μl of the solubilized crystal violet solution was transferred to a new flat bottom plate and absorbance was taken at 550 nm on a microplate spectrophotometer with acetic acid in water as blank.

Metabolic activity (XTT Assay): Biofilms of MRSA, A. baumannii, and P. aeruginosa were grown on 96-well plates as described above. It was washed 3 times with 1×PBS to remove unattached cells. It was incubated with different concentrations of MoSe₂/PLL/F77 solution at 37° C. for 4 h. After incubation the MoSe₂/PLL/F77 solution was discarded and the 96-well plate was washed with 1×PBS. The plate was then incubated with a 150 μl of 1 mg ml⁻¹ solution of XTT salt and 12 μl of 1 mM menadione salt at 37° C. for 5 hours in dark. The solution from each well was taken individually and centrifuged at 2500 rcf using an Eppendorf 5424 Microcentrifuge to remove any nanomaterial particle or biomass that might have been present in the solution. 100 μl of the supernatant was transferred to a new plate and the absorbance was measured at 490 nm on a microplate spectrophotometer.

Confocal imaging: For biofilm visualization by confocal laser scanning microscopy (Nikon C2). Biofilms of MRSA, A. baumannii, and P. aeruginosa were grown for 48 hours (h) on 8 well μ-slides from ibidi. They were incubated with 150 μg ml⁻¹ of MoSe₂/PLL/F77 for 6 h. The stains SYTO9 and propidium iodide (PI) from a LIVE/DEAD BacLight bacterial viability kit were added to treated and untreated (control) biofilms individually. The stained cells were incubated at room for 30 mins followed by imaging.

Biofilm treatment for scanning electron microscope (SEM): Biofilms were grown on stubs of MBEC assay plates as mentioned above with gentle shaking at 110 rpm. The stubs were then incubated in a 150 μg ml⁻¹ concentration of MoSe₂/PLL/F77 solution for the treated samples and 1×PBS for the untreated control sample at 37° C. for 4 h. After the incubation, each individual stub was detached from the plate and fixed in 2.5% glutaraldehyde in DPBS for 12 h and kept at 4° C.

SEM imaging: After fixing the samples in suspension with 2.5% glutaraldehyde in DPBS overnight at 4° C., followed by washing in DPBS three times to remove excess cells. The MBEC stubs were then transferred to a solution of 1% osmium tetroxide in DPBS for 30 mins, followed by thorough washing with deionized water. Samples were dehydrated in a graded ethanol series and critical-point dried in a Balzers-Union CPD-020 unit using carbon dioxide as the transition fluid. After mounting on aluminum stubs, samples were sputter-coated with 10-12 nm of gold-palladium in a Technics Hummer-II unit. Images were generated on a JEOL JSM6300 SEM operated at 15 kV and acquired with an IXRF model 500 digital processor.

Biofilm growth on pre-coated substrates: The MBEC assay plate was dipped in 300 μl (2×MBEC concentration) of MoSe₂/PLL/F77 solution for the coated stubs and 1×PBS for the untreated or control stub at 37° C. for 24-48 h. The solution was allowed to dry out, resulting into a uniform coating around the MBEC stubs. It was them washed 3 times in 1×PBS to remove any excess nanomaterial. Then biofilm was grown following the usual protocol as mentioned above. After growing biofilm, it was then washed in 1×PBS and fixed with the help of glutaraldehyde in DPBS. Another set of samples was made by dipping the MBEC stubs in 100 μl of the MoSe₂/PLL/F77 to ensure coating of approximately half of the stub, followed by biofilm growth on the entire of the stub. This experiment was performed to demonstrate inhibition of biofilm growth by MoSe₂/PLL/F77 coating as well as biofilm growth on the same stub around the untreated area. These samples were then prepared for SEM imaging mentioned above.

Energy dispersive X-ray (EDX) spectroscopy analysis: The half MoSe₂/PLL/F77 coated stubs of MBEC assay plates with biofilm grown on them were prepared for SEM imaging as mentioned above. EDX was carried out with the help of SEM-XL30 Environmental FEG-FEI with accelerating voltage at 20 kV and spot size of 3.

Biofilm growth on coated samples: Hydrophilic poly-tetrafluoroethylene (PTFE) films; poly(methyl methacrylate) (PMMA) coated coverslips, which was obtained by spin coating glass coverslips with PMMA five times at 1500 rpm for 60 s; and medical grade titanium alloy (Ti-alloy) cubes were dipped in 300 μl of MoSe₂/PLL/F77 solution. 1×PBS was used for the uncoated or control samples. The samples were incubated at 37° C. for 24 h until completely dried out. These coated and uncoated samples were washed three times with 1×PBS followed by biofilm growth as mentioned above. They were then fixed in 2.5% glutaraldehyde in DPBS and prepared for SEM imaging.

Biocompatibility of coatings: The cytotoxicity of MoSe₂/PLL/F77 coatings toward HEK 293 cells was evaluated by alamarBlue assay and LDH assay. Cells were seeded in 24-well microplates at a density of 1×10⁵ cells ml⁻¹ in a 500-μl volume with DMEM medium. After 24 h of cell attachment, the plates were washed with DPBS and the MoSe₂/PLL/F77-coated hydrophilic PTFE films were introduced into the DMEM solution. Cells were incubated for 24 h. The wells were washed 3 times with 1×DPBS to remove any unattached cells. To check the viability of the attached cells, they were incubated with 500 μl of 10% alamarBlue solution in DMEM at 37° C. for 5 h. The fluorescence intensity was measured at 530 nm (excitation) and 590 nm (emission) was measured by using a microplate reader. To determine the cytotoxicity of MoSe₂/PLL/F77 through damaged cells, supernatant from each well were pipetted out into a 96-well plate for the LDH assay. 50 μl of supernatant and 50 μl of reaction mixture were incubated at room temperature for 4 h. The absorbance was measured at 490 nm and 680 nm using a microplate reader. Cell damage was expressed as a relative absorbance relative to that of Triton X 100 as a negative control and DMEM medium as a positive control.

1. A biocompatible polymer-coated transition metal chalcogenide (TMC) nanomaterial, comprising a two-dimensional dispersion of TMC at least partially coated with a biocompatible polymer.

2. The biocompatible polymer-coated TMC nanomaterial of claim 1, wherein the TMC is a transition metal dichalcogenide, transition metal monochalcogenide, transition metal trichalcogenide, post-transition metal monochalcogenide, or post-transition metal trichalcogenide.

3. The biocompatible polymer-coated TMC nanomaterial of claim 2, wherein the TMC is selected from MoS₂, MoSe₂, WS₂, WSe₂, Bi₂Se₃, Bi₂S₃, Bi₂Te₃, Sb₂Se₃, Sb₂S₃, Sb₂Te₃, SnSe, and SnS.

4. The biocompatible polymer-coated TMC nanomaterial of claim 1, wherein the biocompatible polymer is selected from a single-stranded DNA (ssDNA), a single-stranded RNA (ssRNA), peptide, poly-L-lysine, poly-D-lysine, Pluronic polymers, Tetronic polymers, and chitosan, or a combination thereof.

5. A method for inhibiting microbiological growth on, or in, a medium which comprises contacting the medium with a biocompatible polymer-coated TMC nanomaterial according to claim 1.

6. The method of claim 5, wherein contacting the medium with the biocompatible polymer-coated TMC nanomaterial inhibits growth of one or more multidrug resistant (MDR) microbial organisms.

7. A method for preparing a biocompatible polymer-coated transition metal chalcogenides (TMC), the method comprising
(a) ultrasonicating a bulk TMDC powder in an aqueous solutions comprising a biocompatible polymer, whereby an ultrasonicated TMDC solution is obtained;
(b) centrifuging the ultrasonicated TMDC solution, whereby a supernatant and a precipitate are obtained; and
(c) collecting the supernatant which comprises a two-dimensional dispersion of biocompatible polymer-coated TMDC.

8. The method of claim 7, wherein the TMC is a transition metal dichalcogenide, transition metal monochalcogenide, transition metal trichalcogenide, post-transition metal monochalcogenide, or post-transition metal trichalcogenide.

9. The method of claim 7, wherein the biocompatible polymer is selected from a single-stranded DNA (ssDNA), a single-stranded RNA (ssRNA), peptide, poly-L-lysine, poly-D-lysine, Pluronic polymers, Tetronic polymers, and chitosan, or a combination thereof.

10. The method of claim 7, wherein the biocompatible polymer is ssDNA or ssRNA having a length of 10 nucleotides to 80 nucleotides.

11. The method of claim 10, wherein the ssDNA has a nucleotide sequence comprising at least ten repeats of GT (GT₁₀).

12. The method of claim 10, wherein the ssDNA has a nucleotide sequence comprising 10-20 consecutive thymidine (T) bases.

13. A method for inhibiting microbiological growth on, or in, a medium which comprises coating the medium with a biocompatible polymer-coated TMC nanomaterial prepared according to claim 7.

14. The method of claim 13, wherein the medium is a medical device.

15. The method of claim 13, wherein coating the medium with the biocompatible polymer coated TMC nanomaterial inhibits growth of one or more multidrug resistant (MDR) microbial organisms.


SEQUENCE LISTING (8 sequences)

SEQ ID NO: 1 - DNA, 20 bp, Artificial Sequence
  synthetic
gtgtgtgtgtgtgtgtgtgt

SEQ ID NO: 2 - DNA, 20 bp, Artificial Sequence
  synthetic
tttttttttttttttttttt

SEQ ID NO: 3 - DNA, 20 bp, Artificial Sequence
  synthetic
cacacacacacacacacaca

SEQ ID NO: 4 - DNA, 20 bp, Artificial Sequence
  synthetic
aaaaaaaaaaaaaaaaaaaa

SEQ ID NO: 5 - DNA, 20 bp, Artificial Sequence
  synthetic
cccccccccccccccccccc

SEQ ID NO: 6 - DNA, 20 bp, Artificial Sequence
  synthetic
ggggaggggaggggagggga

SEQ ID NO: 7 - DNA, 20 bp, Artificial Sequence
  synthetic
ggggtggggtggggtggggt

SEQ ID NO: 8 - DNA, 10 bp, Artificial Sequence
  synthetic
gtgtgtgtgt